(19) United States
(12) Patent Application Publication
(10) Pub. No.: US 2005/0214748 A1
(43) Pub. Date: Sep. 29, 2005

(54) Peptide-based diagnostic reagents for SARS

(76) Inventors: Chang Yi Wang, Cold Spring Harbor, NY (US); Xinde Fang, Fresh Meadows, NY (US); Tseng Yuan Chang, West Islip, NY (US); Scott Liu, Lake Grove, NY (US); Shugene Lynn, Taoyuan (TW); Charles Sia, North York (CA)

Correspondence Address: MORGAN & FINNEGAN, L.L.P., 3 WORLD FINANCIAL CENTER, NEW YORK, NY, 10281-2101, US

(21) Appl. No.: 10/983,854

(22) Filed: Nov. 8, 2004

Related U.S. Application Data
(63) Continuation-in-part of application No. 10/712,812, filed on Nov. 12, 2003.

(51) Int. Cl. C12Q 1/70

(52) U.S. Cl. 435/5

(57) ABSTRACT

The present invention is directed to antigenic peptides and peptide compositions selected from the Membrane glycoprotein (M), the Spike glycoprotein (S), and the Nucleocapsid (N) protein antigens of the SARS coronavirus (SCoV). The present invention is also directed to methods of use of the peptides of the invention, e.g., for the detection of SARS-associated antibodies. Detection methods include enzyme-linked immunosorbent assay (ELISA) or other immunoassay procedures.

This application is a continuation-in-part application of co-pending application Ser. No. 10/712,812, filed Nov. 12, 2003, the contents of which is incorporated in its entirety.

### FIELD OF THE INVENTION

Severe acute respiratory syndrome (SARS) is a recently discovered atypical pneumonia that has been spreading throughout the world. The illness originated in the Guangdong province of China in November 2002 and as of May 2003 areas of local transmission had appeared in Beijing, Inner Mongolia, Shanxi, Hebei, and Tianjin regions of China, in Hong Kong, Taiwan, Mongolia, Philippines, Singapore, Viet Nam, and Canada. Cases have been reported to the World Health Organization (WHO) in 31 countries in all, on five continents¹. (The superscript numbers refer to publications, which more fully describe the state of the art to which this invention pertains. The disclosures of these references are hereby incorporated by reference. The citation of each reference is found at the end of the Background Of The Invention section). SARS has the general features of starting with a fever greater than 38° C., headache, and sore throat. The incubation period for the disease is usually from 2 to 7 days, and the patients could develop a dry, nonproductive cough and shortness of breath. Death from progressive respiratory failure occurs in about 3-8% cases. Given these possible symptoms, variable patterns of reactions to the infection are usually found among SARS infected individuals 2. The recent isolation of coronaviruses from multiple SARS patients, the culturing and characterization of the virus, and the transmission of disease by the virus to macaques has fulfilled Koch's postulates and established SARS coronavirus (SCoV) as the agent causing the disease³. The development of safe and readily available serologic tests for SARS, based on non-biohazardous synthetic peptide antigens, will be of significant value for the control and elimination of SCoV.

### BACKGROUND OF THE INVENTION

Serologic testing for infection by SCoV has been developed using enzyme-linked immunosorbant assays (ELISA) or indirect immunofluorescence assays (IFA) based on using the whole virus as the antigen. These immunoassays detect antibodies in the mid and late stages of SARS^{4,5}, and using these whole virus antigens SCoV-specific IgG has been found to persist through week 12 post-infection⁶. Thus, serologic assays have been proven to be useful both for the diagnosis and for the epidemiological surveillance of SARS.

The major structural proteins common to coronaviruses include the spike (S), membrane (M), and nucleocapsid (N). The S, M, and N proteins are antigens that contribute to generating the host immune response⁷. Because of the low level of similarity between the predicted amino acid sequences of SCoV and other coronaviruses, comparisons between SARS coronavirus and other coronaviruses, including the 229E human coronavirus, for primary amino acid sequences does not provide insight into the antigenic sites of SCoV⁸⁻¹⁰. Accordingly, new studies are required to identify specific SCoV antigens. For example, Spike glycoprotein (S) has been well-characterized as the surface antigen of viruses of the family Coronaviridae that binds to the host cell receptor. The surface protein is well known to be a target for neutralizing humoral immune responses and such responses are associated with protective immunity⁸. Therefore, antibodies against the SCoV S glycoprotein in SARS patients may have a protective value. SCoV S antigens expressed by recombinant Escherichia coli as intact S or as large segments are being studied as serological reagents¹¹. The membrane glycoproteins (M) of coronaviruses are also prominently surface exposed and are, in general, expected to contain immunodominant epitopes that may be useful for immunoassays⁷. For example, the M protein of canine coronavirus was cloned and expressed in Escherichia coli and the recombinant antigen was found to be useful in an ELISA for the detection of antibodies against the canine coronavirus in dog sera¹². However, the effectiveness of the M glycoprotein of the SARS coronavirus as a diagnostic reagent remains to be demonstrated. The N protein also can be a useful subunit antigen for the detection of anti-SCoV antibodies in patient sera and has been expressed as recombinant antigens^{13,14}.

However, the cloning of entire proteins of the SCoV is complicated and involves the use of hazardous materials as does the immunoassay methods employing whole SCoV antigen. It is desirable to utilize the advantages of synthetic peptides over complex biological materials such as viral extracts or recombinant proteins, for the immunosorbent in immunoassays. No biohazardous materials are used in the manufacture of synthetic peptides. The peptides are defined molecules that can be easily manufactured by a reproducible chemical process. The quality can be controlled and as a result, reproducibility of the test results can be assured. With site-specific epitopes the signal-to-noise ratio is boosted and sensitivity is heightened, while specificity is optimized due to the reduction of undesirable cross-reactive epitopes. Moreover, the use of synthetic peptides eliminates the false-positive results caused by the presence of antigenic materials originating from host cells and from recombinant protein expression systems that may be co-purified with SCoV viral and recombinant proteins. The high specificity of a synthetic peptide-based immunoassay makes it useful for differentiating infections caused by different viruses having similar clinical symptoms, e.g., RSV or influenza versus SARS. The costs for producing immunoassays having synthetic peptides are relatively low in comparison to tests using virally and recombinantly-produced antigens because very small amounts of peptide are required for each test procedure and the expense of synthesizing peptides is relatively low compared to the expense of producing biologicals¹⁵⁻¹⁷. With regard to the diagnosis of SARS, the apparent genetic stability of SCoV and the conservation of epitopes across isolates⁹, favors an approach using controlled and well-defined antigens rather than complex antigens.

At present, it is difficult to produce synthetic peptide-based immunoassays. A number of algorithms have been developed to predict candidate epitopes in proteins from the amino acid sequence, but, the present state of knowledge of protein structure does not enable the precise prediction of the amino acid sequences that represent highly antigenic epitopes. Thus, the usefulness of a synthetic peptide as an antigen must be empirically established. We have used serological analysis to map epitopes within the protein antigens of infectious viruses, as an empirical approach. These include the identification of epitopes of HIV¹⁸, HCV^{19,20}, HTLV I and II²¹, and foot-and-mouth disease virus (FMDV)²².

The development of safe, rapid, reliable, easy-to-use and readily available serologic tests for SARS, based on non-biohazardous synthetic peptide antigens, is of significant value. Such tests can be used in diagnostics and in epidemiological surveys for the control and eventual elimination of SARS.

### REFERENCES

1. Severe Acute Respiratory Disease: World Health Organization www.who.int/csr/sars/en.
2. CDC. Updated interim surveillance case definition for severe acute respiratory syndrome (SARS)—United States, Apr. 29, 2003. MMWR 2003; 52:391-393.
3. Fouchier R A M, Kuiken T, Schutten M, et al., Koch's postulates fulfilled for SARS virus. Nature 2003; 423:240.
4. Peiris J S M, Lai S T, Poon L L M, et al., Coronavirus as a possible cause of severe acute respiratory syndrome. Lancet 2003; 361:1319-1325.
5. National Research Project for SARS Beijing Group. Serum antibodies detection for serological diagnosis of severe acute respiratory syndrome. Zhonghua Jie He He Hu Xi Za Zhi 2003; 26:339-342.
6. Li G, Chen X, Xu A, Profile of specific antibodies to the SARS-associated coronavirus. N Eng J Med 2003; 349:508-509.
7. Holmes K V, Chapter 29. Coronaviridae and their replication. In: Fields B N, Knipe D M, eds. Virology 2nd edition. Vol. 1. New York: Raven Press, 1990:841-864.
8. Rota P A, Oberste M S, Monroe S S, et al., Characterization of a novel coronavirus associated with severe acute respiratory syndrome. Science 2003; 300:1394-1398.
9. Ruan Y, Wei C L, Ee L A, et al., Comparative full-length genome sequence analysis of 14 SARS coronavirus isolates and common mutations associated with putative origins of infection. Lancet 2003; 361:1779-1785.
10. Wang Y, Xie Y, Chen W, Immunoinformatic analysis for the epitopes on SARS virus surface protein. Beijing Da Xue Xue Bao 2003; 35:70-71.
11. Yu H, Yang Y, Zhang W, et al., Expression and purification of recombinant SARS coronavirus spike protein. Sheng Wu Hua Xue Yu Sheng Wu Wu Li Xue Bao (Shanghai) 2003; 35:774-778.
12. Elia G, Fiermonte G, Prattelli A, et al., Recombinant M protein-based ELISA test for detection of antibodies to canine coronavirus. J Virol Methods 2003; 109:139-142.
13. Che X Y, Hao W, Qiu L W, et al., Antibody response of patients with severe acute respiratory syndrome (SARS) to nucleocapsid antigen of SARS-associated coronavirus. Di Yi Jun Yi Da Xue Xue Bao 2003; 23:637-639.
14. Krokhin 0, Li Y, Andonov A, et al., Mass spectrometric characterization of proteins from the SARS virus: A preliminary report. Mol Cell Proteomics 2003; 2:346-356.
15. Wang C Y, Synthetic peptide-based immunodiagnosis of retrovirus infections: Current status and future prospects. Synthetic peptides in biotechnology: Alan R. Liss, Inc., 1988:131-148.
16. Wang C Y, Hosein B, Synthetic peptides specific for the detection of antibodies to HCV, diagnosis of HCV infection and preventions thereof as vaccines. U.S. Pat. No. 5,747,239 1998.
17. Wang C Y, Peptides derived from the non-structural proteins of foot and mouth disease virus as diagnostic reagents. U.S. Pat. No. 6,048,538 2000.
18. Wang J J G, Steel S, Wisniewolski R, Wang C Y, Detection of antibodies to human T-lymphotropic virus type III by using a synthetic peptide of 21 amino acid residues corresponding to a highly antigenic segment of gp41 envelope protein. Proc Nat Acad Sci USA 1986; 83:6159-6163.
19. Hosein B, Fang C T, Popovsky M A, Ye J, Zhang M L, Wang C Y, Improved serodiagnosis of hepatitis C virus infection with synthetic peptide antigen from capsid protein. Proc Nat Acad Sci USA 1991; 88:3647-3651.
20. Wang C Y, Hosein B, Linear and branched peptides effective in diagnosing and detecting non-A, non-B hepatitis. U.S. Pat. No. 5,639,594 1997.
21. Wang C Y, Synthetic peptide compositions with immunoreactivities to antibodies to HTLV. U.S. Pat. No. 5,681,696 1997.
22. Shen F, Chen P D, Walfield A M, et al., Differentiation of convalescent animals from those vaccinated against foot-and-mouth disease by a peptide ELISA. Vaccine 1999; 17:3039-3049.
23. Marra M A, Jones S J M, Astell C R, et al., The genome sequence of the SARS-associated coronavirus. Science 2003; 300:1399-1404.
24. Merrifield R B, Solid phase synthesis. Science 1986; 232:341-347.
25. Fields G B, Tian Z, Barany G, Chapter 3. Principles and practice of solid-phase peptide synthesis. In: Grant G A, ed. Synthetic Peptides. A User's Guide. New York, N.Y.: W.H. Freeman & Company, 1992:77-183.
26. Harlow E, Lane D, Chapter 14. Immunoassays. Antibodies A Laboratory Manual. Plainview, N.Y.: Cold Spring Harbor Laboratory Press, 1988:553-612.
27. Flaherty R J, Evidence-based medicine for student health services. 2003: Montana State University, Bozeman.:
   http://www.montana.edu/wwwebm/URI.htm.

### BRIEF DESCRIPTION OF THE INVENTION

The present invention relates to SCoV peptides where one or more of these peptides, or segments thereof, is used as antigenic compositions for immunological applications. Such applications include use in immunoassays and/or diagnostic kits as the solid phase immunosorbent and use for the design of a SARS vaccine where such peptides may represent protective B cell epitopes. Immunoassays and/or diagnostic kits containing one or more of these peptides, or segments thereof, are useful to identify antibodies induced by infection or by vaccination. Such tests can be used to screen for the presence of SCoV infection in the clinic, for epidemiological surveillance, and for testing the efficacy of vaccines.

One aspect of the peptides and peptide compositions of the invention is to provide a method of diagnosing SCoV infection in a patient, comprising the steps of:
i. contacting the patient sample with one or more SCoV antigenic peptides or immunologically functional analogues thereof under conditions conducive to binding; and
ii. measuring binding between said patient sample and said SCoV antigenic peptides or immunologically functional analogues thereof,
   wherein detection of binding between said patient sample and said SCoV antigenic peptides or immunologically functional analogues thereof indicates the presence of anti-SCoV antibodies (and therefore SCoV) in said patient sample.

According to the present invention, a series of synthetic peptides representing immunoreactive regions of the SCoV spike (S) (peptides 3180b, 3180c, 3180, 3K3180c), membrane (M) (peptides 3301, 3K3301), and nucleocapsid (N) (peptides 3187b, 3187, 3189a, 3189b, 3189, 3190a, 3190b, 3K3190b, 3190) proteins each described by specific sequences, SEQ ID NOS. 1-4,5-6, and 7-15 for S, M, and N respectively, have been identified and made by solid phase peptide synthesis. These peptides have been found to be useful for the detection of antibodies to SCoV in sera and body fluids and for the diagnosis of SARS. In addition, according to the present invention, mixtures of these peptides may be used to detect the presence of antibodies to SCoV in sera and other body fluids.

According to the present invention, a peptide composition useful as a reagent in immunoassays for the detection of antibodies to SCoV and diagnosis of SARS may be selected from a peptide from the S protein (SEQ ID NOS. 1-4), a peptide from the M protein (SEQ ID NOS. 5-6), a peptide of the N protein (SEQ ID NOS. 7-16), or a mixture thereof. (These sequences were adopted from the Tor2 isolate of SCoV²³, made available in Entrez Genomes under RefSeq accession NC_{—}004718 and assigned GenBank accession no. AY274119, and are localized on the SCoV genome in FIGS. 2 and 3). Useful compositions are represented by the amino acid sequences shown in Table 1 as well as immunologically functional analogues, mixtures, conjugates and polymers thereof.

Another aspect of these peptide compositions allows for the development of chemically synthesized immunoassay reagents that can be readily quality controlled and used to develop sensitive and accurate methods for monitoring SCoV infection.

A further aspect of the present invention also provides immunoassay test kits for the detection and diagnosis of SARS by using an antigenically effective amount of the subject peptide composition as the solid phase immunosorbent in said test kits. The preferred immunoassay test kit format is ELISA. These immunoassays can be used as diagnostic tools to screen for the presence of SCoV infection in the clinic, to monitor antibody and viral antigen expression during SCoV infection and thereby determine correlations between the presence of specific antibodies and the prognosis of SARS in patients, and in epidemiological surveys and/or to monitor the effectiveness of a vaccination program.

### BRIEF DESCRIPTIONS OF THE DRAWINGS

FIG. 1. Genomic structure of SARS coronavirus Tor2, in comparison to that of human coronavirus 229E, and the deduced structural protein sequences of Tor2, based on Entrez Genomes RefSeq accession NC_{—}004718, also GenBank accession no. AY274119.

FIG. 2. Location of the putative M and N protein encoding sequences on the SCoV Tor2 genome and overlapping peptides, with amino acid positions, for mapping candidate antigenic epitopes found in M and N proteins.

FIG. 3. Location of the putative S protein encoding sequence on the SCoV Tor2 genome and overlapping peptides, with amino acid positions, for mapping candidate antigenic epitopes found in S protein.

FIG. 4. Distribution of antibody reactivities to antigenic S and M peptides in sera from 672 random blood donors taken from a zero seroprevalence population. Samples were tested by the 3180c+3301 mixed peptide SCoV ELISA. Reactivities are shown as Signal/Cutoff ratios (S/C). The mean absorbance (A₄₅₀) for the donor sera was 0.074±0.034. The cutoff value for the mixed peptide ELISA is set as the mean A450±6 standard deviations.

### DETAILED DESCRIPTION OF THE INVENTION

The invention provides SCoV antigenic peptides. SCoV antigenic peptides are peptides that correspond to antigenic sites of SCoV proteins. Such peptides correspond to the portion of the amino acid sequence of naturally occurring SCoV that forms an epitope for antibody recognition. In a preferred embodiment, the epitope that forms the SCoV antigenic peptide is an epitope that is typically recognized in a SARS infection wherein the SARS-infected patient produces antibodies to the epitope. Such epitopes can be empirically determined using samples from SARS-infected patients known to be infected with SCoV. Any immunoassay known in the art, e.g. ELISA, immunodot, immunoblot, etc., can be used to determine if antibodies are present in a sample from an SCoV-infected patient that bind to a particular fragment of a SCoV protein. In a specific embodiment, SCoV antigenic peptides are those peptides that correspond to the antigenic sites of S, M, or N proteins of SCoV. The SCoV antigenic peptides of the invention can vary in length from about 15 to about 100 amino acid residues. Preferably the SCoV antigenic peptides of the invention are about 20 to about 85 amino acid residues. In preferred embodiments, SCoV antigenic peptides are selected from the group consisting of SEQ ID NOS: 1, 5, 7, 9, and 12. The production and use of the SCoV antigenic peptides of the invention are within the scope of the present invention.

The invention further provides immunologically functional analogues of SCoV antigenic peptides. An immunologically functional analogue has been modified when compared to the corresponding of SCoV antigenic peptide in some way (e.g., change in sequence or charge, covalent attachment to another moiety, addition of one or more branched structures, and/or multimerization) yet retains substantially the same secondary and tertiary structure and/or immunogenicity as the original SCoV antigenic peptide. Thus antibodies that bind to a particular SCoV antigenic peptide will also bind to the immunologically functional analogue of that SCoV antigenic peptide with substantially similar efficacy. In preferred embodiments, immunologically functional analogues of SCoV antigenic peptides are selected from the group consisting of SEQ ID NOS: 2, 3, 4, 6, 8, 10, 11, 13, 14, and 15. The production and use of such peptide analogues are within the scope of the present invention.

In one embodiment, immunologically functional analogues of SCoV antigenic peptides are meant to encompass of SCoV antigenic peptides that have been modified by sequence substitutions, additions, or deletions. In a specific embodiment, immunologically functional analogues can be modified by sequence substitutions that are conservative. Conservative substitutions are when one amino acid residue is substituted for another amino acid residue with similar chemical properties. For example, the nonpolar (hydrophobic) amino acids include alanine, leucine, isoleucine, valine, proline, phenylalanine, tryptophan and methionine; the polar neutral amino acids include glycine, serine, threonine, cysteine, tyrosine, asparagine, and glutamine; the positively charged (basic) amino acids include arginine, lysine and histidine; and the negatively charged (acidic) amino acids include aspartic acid and glutamic acid. Non-conservative substitutions are when one amino acid residue is substituted for another amino acid residue that different chemical properties. Substituted residues can be either classical or non-classical amino acids. As used herein, classical amino acids are the 20 amino acids commonly found in proteins (i.e., alanine, aspartic acid, asparagine, arginine, cysteine, glycine, glutamine, glutamic acid, histidine, isoleucine, leucine, lysine, methionine, phenylalanine, proline, serine, threonine, tyrosine, tryptophan and valine) and include both the D- and L-forms of such amino acids. As used herein non-classical amino acids include both D- and L-forms of any other amino acids that can be incorporated into a protein or peptide, whether found in nature or whether synthetically produced. Non-classical amino acids include, but are not limited to, β-alanine, ornithine, norleucine, norvaline, hydroxyproline, thyroxine, gamma-amino butyric acid, homoserine, citrulline, α-amino isobutyric acid, 4-aminobutyric acid, sarcosine, cysteic acid, t-butylglycine, t-butylalanine, phenylglycine, cyclohexylalanine, and the like.

In another specific embodiment, immunologically functional analogues can be modified by amino acid additions to the N-terminus, C-terminus, and/or middle of the peptide. In preferred embodiments, additions are to the N-terminus or C-terminus of the peptide. Additions can be of 1, 2, 3, 4, 5, 6, 7, 8, 9, 10, 11, 12, 13, 14, 15, 16, 17, 18, 19, or 20 amino acid resides. Such additions may constitute amino acid sequences that are present in SCoV in their entirety or in part. In a preferred embodiment, additions of amino acid sequences that are present in SCoV are of 15 amino acids or less. Such additions may also constitute amino acid sequences which are not present in SCoV. Addition of sequences which are not present in SCoV include, but are not limited to, small charged sequences (e.g., lysine-lysine-lysine) and sequences that enable the formation of branched structures (e.g., lysine or methionine). In a preferred embodiment, additions of amino acid sequences that are not present in SCoV are of 5 amino acids or less. Residue additions can be either classical or non-classical amino acids or a mixture thereof.

In another specific embodiment, immunologically functional analogues can be modified by amino acid deletions to the N-terminus, C-terminus, and/or middle of the peptide. In preferred embodiments, deletions are to the N-terminus or C-terminus of the peptide. Deletions can be of 1, 2, 3, 4, 5, 6, 7, 8, 9, 10, 11, 12, 13, 14, 15, 16, 17, 18, 19, or 20 amino acid resides. In a preferred embodiment, deletions of amino acid sequences are of 10 amino acids or less.

In another embodiment, immunologically functional analogues of SCoV antigenic peptides are meant to encompass SCoV antigenic peptides that have been modified by covalent attachment to another moiety. The covalent attachment between the peptide and moiety may be direct or indirect (e.g. through a linker molecule) by methods known in the art. In a specific embodiment, the moiety is a carrier molecule (e.g. bovine serum albumin or human serum albumin). In another specific embodiment, the moiety is a red blood cell. In another specific embodiment, the moiety is a latex particle. In another specific embodiment, the moiety is a bead.

In another embodiment, immunologically functional analogues of SCoV antigenic peptides are meant to encompass SCoV antigenic peptides that have been modified by an alteration in charge. Such alteration in charge may be the result of amino acid substitutions, additions, or deletions, or the covalent attachment of a charged molecule. The alteration in charge may have the result of making the peptide more basic, more acidic, or more neutral as compared to the unmodified peptide. In a preferred embodiment, the peptide is made more basic by the addition of 1-5 lysine residues to the N-terminus or C-terminus. In a more preferred embodiment, the peptide is made more basic by the addition of 3 lysine residues to the N-terminus. In a most preferred embodiment, the immunologically functional analogue of a SCoV antigenic peptide with a altered charge such that is more basic is selected from the group consisting of 3K3180c, 3K3301, and 3K3190b (SEQ ID NOS. 4, 6, and 14, respectively).

In another embodiment, immunologically functional analogues of SCoV antigenic peptides are meant to encompass SCoV antigenic peptides that have been modified by addition of one or more branched structures. The branched peptides of the present invention are represented by one of the formulae:
(peptide)₂X
(peptide)₄X₂X
(peptide)₈X₄X₂X
(peptide)₁₆X₈X₄X₂X
   wherein X is an amino acid or an amino acid (either classical or non-classical amino acids) having two amino groups and one carboxyl group, each group capable of forming a peptide bond linkage. In a preferred embodiment, X is lysine or methionine or non-classical amino acid residue analogue thereof. In a more preferred embodiment, X is lysine or a non classical amino acid analogue thereof (e.g., ornithine). Branched peptides of the invention include, but are not limited to, dimers, tetramers, octamers, and hexadecamers. In contrast, the linear peptides of this invention are represented by the formula
   (peptide)-Y
   wherein Y is —OH or —NH₂.

In another embodiment, immunologically functional analogues of SCoV antigenic peptides are meant to encompass SCoV antigenic peptides that have been modified by multimerization. Such multimers may be linear or branched multimers. The individual peptides may be linked directly (e.g., as a fusion protein for linear multimers or with the addition of an amino acid residue having two amino groups and one carboxyl group for branched multimers) or indirectly (e.g., through the use of a linker molecule). Such multimers comprise at least two peptides and such peptides may or may not be identical to each other (e.g., multimers may comprise different SCoV antigenic peptides, different immunologically functional analogues of SCoV antigenic peptides, or a mixture of both).

In another embodiment, immunologically functional analogues of SCoV antigenic peptides are meant to encompass SCoV antigenic peptides that have been modified to include amino acid sequences from strains of SCoV other than Tor2. Such peptides contain substitutions (conservative or non-conservative), additions, and/or deletions such that regions of SCoV proteins from SCoV strains whose sequences vary from those of SCoV Tor2 are represented. Thus, strain-to-strain variation among different isolates of SCoV can be accommodated.

In another embodiment, immunologically functional analogues of SCoV antigenic peptides are meant to encompass SCoV antigenic peptides that have been modified by chemical modification, e.g., by glycosylation, acetylation, phosphorylation, amidation, derivatization by known protecting/blocking groups, proteolytic cleavage, etc. Any of numerous chemical modifications may be carried out by known techniques, including but not limited to, reagents useful for protection or modification of free NH2-groups, free COOH— groups, OH— groups, side groups of Trp-, Tyr-, Phe-, His-, Arg-, or Lys-; specific chemical cleavage by cyanogen bromide, hydroxylamine, BNPS-Skatole, acid, or alkali hydrolysis; enzymatic cleavage by trypsin, chymotrypsin, papain, V8 protease, NaBH₄; acetylation, formylation, oxidation, reduction; metabolic synthesis in the presence of tunicamycin; etc.

The present invention encompasses compositions of SCoV antigenic peptides, compositions of and immunologically functional analogues of SCoV antigenic peptides, and uses thereof. In embodiment, compositions of the invention may be a pure composition comprising only one type of SCoV antigenic peptide or immunologically functional analogue of an SCoV antigenic peptide. In another embodiment, compositions of the invention may be mixed compositions comprising more than one type of SCoV antigenic peptide (e.g., more than one antigenic peptide from the same protein, different proteins, or a mixture of both) or immunologically functional analogue of an SCoV antigenic peptide or a mixture of both SCoV antigenic peptides and immunologically functional analogues of SCoV antigenic peptides. Such pure or mixed compositions may be used in the methods of the invention. When mixed compositions are used in the methods of the invention (e.g., for diagnosis of SCoV infection or detection of SCoV antibodies) the effective ratio of the peptides can be readily determined by one of ordinary skill in the art. Typically, these ratios range from about 1 to about 50 on a weight basis of peptide.

SCoV antigenic peptides and immunologically functional analogues thereof were isolated by finding antigenic sites of relevant immunogenicity using serological analysis of overlapping synthetic peptides taken from SCoV protein antigens (Tor2 isolate of SCoV²³) with sera from patients with clinically diagnosed SARS. This process of serological validation led to the identification and further definition of SARS-immunoreactive peptides.

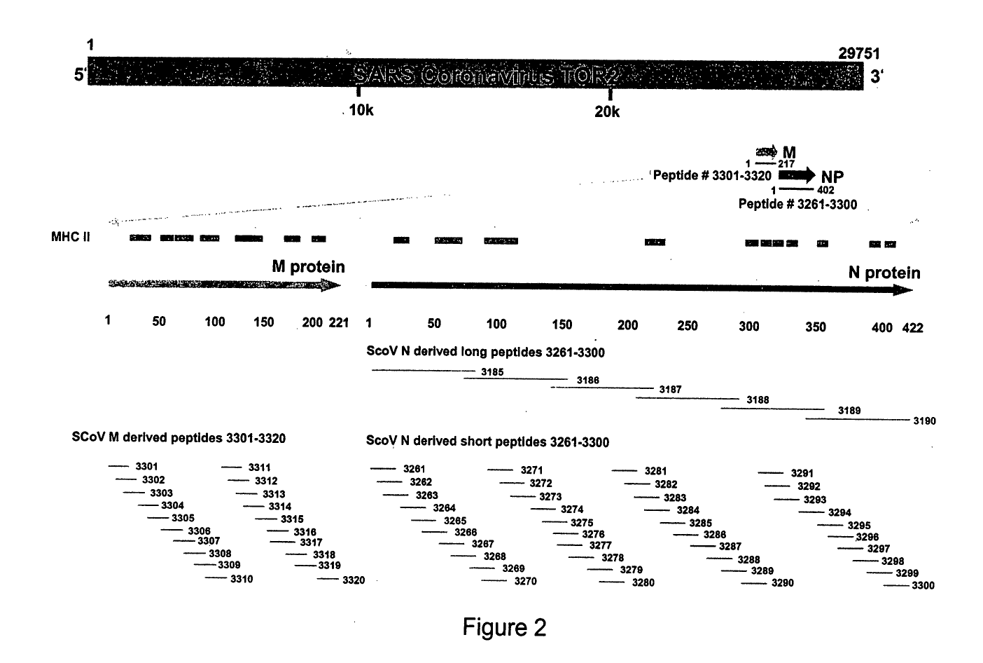

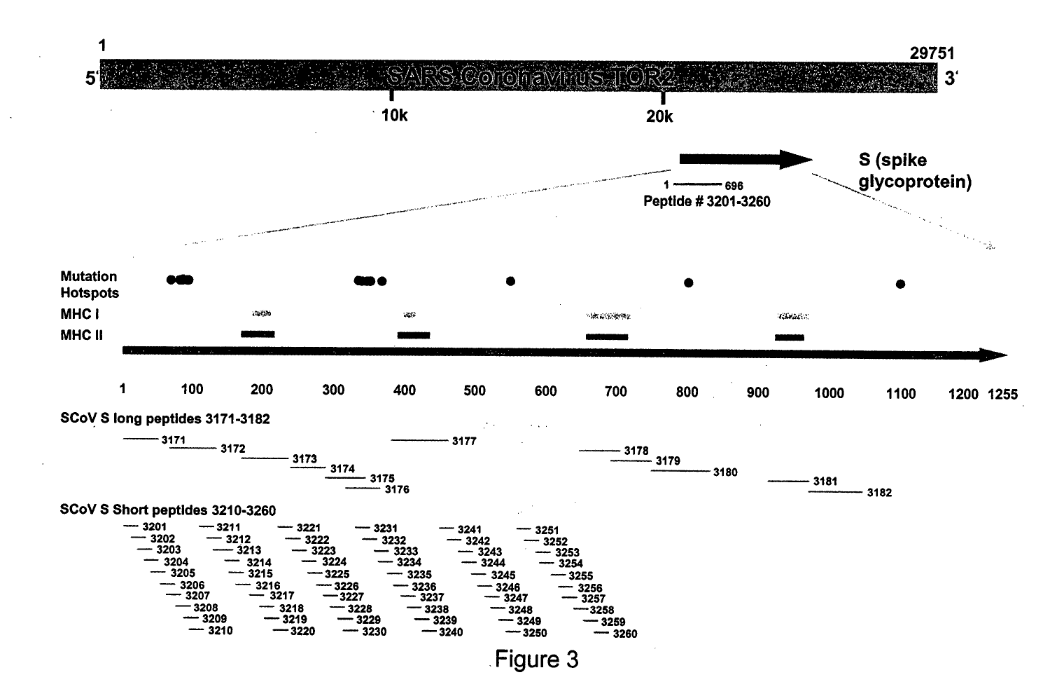

Accordingly, SCoV antigenic peptides and immunologically functional analogues thereof of SEQ ID NOS:1-15 were isolated. In preferred embodiments, peptides of this invention are those of SEQ ID NOS: 2, 4, 5, 6, 13, 14, and 15 (These sequences were taken from the Tor2 isolate of SCoV²³ and are positioned on the SCoV genome in FIGS. 2 and 3). All the peptides of the invention are designated by their respective sequence identification numbers as shown in Table 1.

TABLE 1

Amino acid sequences of SCoV antigenic
peptides derived from S, M, and N proteins

S Protein-Derived Peptides

| | | |
|---|---|---|
| ⁷⁷⁷KYFGGFNFSQILPDPLKPTKRSFIEDLLFNKVTLADAGFMQYGE⁸²¹ | 3180b | (SEQ |
| | | ID No. |
| | | 1) |
| ⁷⁶⁷VKQMYKTPTLKYFGGFNFSQILPDPLKPTKRSFIEDLLFNKVTLADAGFMKQYGE⁸²¹ | 3180c | (SEQ |
| | | ID No. |
| | | 2) |
| KKKVKQMYKTPTLKYFGGFNFSQILPDPLKPTKRSFIEDLLFNKVTLADAGFMKQYGE⁸²¹ | (SEQ | |
| | | ID No. |
| | | 3) |
| ⁷⁵²AAEQDRNTREVFAQVKQMYKTPTLKYFGGFNFSQILPDPLKPTKRSFIEDLLFNKVTLADAGFMKQYGE⁸²¹ | 3180 | (SEQ |
| | | ID No. |
| | | 4) |

M Protein-Derived Peptides

| | | |
|---|---|---|
| ¹MADNGTITVEELKQLLEQWNLV²² | 3301 | (SEQ |
| | | ID No. |
| | | 5) |
| KKK¹MADNGTITVEELKQLLEQWNLV²² | 3K3301 | (SEQ |
| | | ID No. |
| | | 6) |

N Protein-Derived Peptides

| | | |
|---|---|---|
| ¹⁶¹QLPQGTTLPKGFYAEGSRGGGSQASSRSSSRSRGNSRNSTPGSSRGNSPARMASGGGETALALLLL²²⁵ | 3187b | (SEQ |
| | | ID No. |
| | | 7) |
| ¹⁴⁶HIGTRNPNNNAATVLQLPQGTTLPKGFYAEGSRGGSQASSRSSSRSRGNSRNSTPGSSRGNSPARMASGGGETALALLLL²²⁵ | 3187 | (SEQ |
| | | ID No. |
| | | 8) |
| ³⁰⁶AQFAPSASAFFGMSRIGMEVTPSGTWLTYHGAIKLDDKDPQFKDNVILLN³⁵⁵ | 3189a | (SEQ |
| | | ID No. |
| | | 9) |
| ²⁹¹DLIRQGTDYKHWPQIAQFAPSASAFFGMSRIGMEVTPSGTWLTYHGAIKLDDKDPQFKDNVILLN³⁵⁵ | 3189b | (SEQ |
| | | ID No. |
| | | 10) |
| ²⁷⁶GRRGPEQTQGNFGDLIRQGTDYKHWPQIAQFAPSASAFFGMSRIGMEVTPSGTWLTYHGAIKLDDKDPQFKDNVILLN³⁵⁵ | 3189 | (SEQ |
| | | ID No. |
| | | 11) |
| ³⁷¹KKDKKKKTDEAQPLPQRQKKQPTVTLLPAADMDDFSRQLQNSMSGASADSTQ⁴²² | 3190a | (SEQ |
| | | ID No. |
| | | 12) |
| ³⁵⁶KHIDAYKTFPPTEPKKDKKKKTDEAQPLPQRQKKQPTVTLLPAADMDDFSRQLQNSMSGASADSTQ^{3190b} | (SEQ | |
| | | ID No. |
| | | 13) |
| KKK³⁵⁶KHIDAYKTFPPTEPKKDKKKKTDEAQPLPQRQKKQPTVTLLPAADMDDFSRQLQNSMSGASADSTQ⁴²² | (SEQ | |
| | | ID No. |
| | | 14) |
| ³⁴¹DDKDPQFKDNVILLNKHIDAYKTFPPTEPKKDKKKKTDEAQPLPQRQKKQPTVTLLPAADMDDFSRQLQNNSMSGASADSTQ⁴²² | 3190 | (SEQ |
| | | ID No. |
| | | 15) |

To identify the highly antigenic Spike (S) peptides (SEQ ID NOS 1-4), more than 100 overlapping peptides with lengths from 20 to 77 residues were designed, synthesized and tested with a panel of SARS patient and normal human sera. Among the S peptides tested, only four were found to have significant SCoV antigenicity (SEQ ID NOS. 1-4). These include a segment of 54 amino acids (SEQ ID NO: 2, 3180c). A deletion from the N-terminus of 10 amino acids resulted in peptide 3180b (SEQ ID NO: 1) having slightly diminished reactivity from 3180c (SEQ ID NO. 2). Antigenicity was also retained with an extension from the N-terminus of 3180c (SEQ ID NO. 2) of 15 amino acids (SEQ ID NO. 4, 3180) (see Table 6). Therefore, immunologically functional analogues of the subject peptides include extension on their termini by segments from the SCoV antigens of about 15 amino acids and deletions of about 10 amino acids.

By way of a non-limiting example, immunologically functional analogues of the peptides of the invention can have from 1 to about 5 additional amino acids (classical and non-classical) added to the terminal amino acids. For example, the sequence KKK (Lys-Lys-Lys) can be added to the amino terminus of any of these peptides for a change in charge, e.g., 3K3180c, 3K3301, and 3K3190b (SEQ ID NOS. 3, 6, and 14, respectively). A methionine residue can be placed at the carboxyl terminus of the peptide moiety, i.e. between the peptide moiety and a branch structure, to enable the formation of a branched structure.

The peptides of the invention are useful for the detection of SCoV antibodies in patient samples for the diagnosis of SARS. As used herein, a patient sample is meant to encompass any bodily fluid or tissue that may contain antibodies, including, but not limited to, blood, serum, plasma, saliva, urine, mucus, fecal matter, tissue extracts, tissue fluids. As used herein, the term patient is meant to encompass a mammal such as a non-primate (e.g., cow, pig, horse, cat, dog, rat etc.) and a primate (e.g., monkey and human), most preferably a human. The peptides of the invention can be used in immunoassays to detect the presence of anti-SCoV antibodies in the patient sample. Any immunoassay known in the art can be used. For example, the patient sample can be contacted with one or more SCoV antigenic peptides or immunologically functional analogues thereof under conditions conducive to binding. Any binding between said patient sample and said SCoV antigenic peptides or immunologically functional analogues thereof can be measured by methods known in the art. Detection of binding between said patient sample and said SCoV antigenic peptides or immunologically functional analogues thereof indicates the presence of SCoV in said patient sample. In a more specific embodiment, an ELISA immunoassay can be used to assay a patient sample for the presence of anti-SCoV antibodies comprising the steps of:
i. attaching a peptide selected from the group consisting of SEQ ID NOS: 1-4,5-6 and 7-15 to a solid support,
ii. exposing said peptide attached to said solid support to a sample containing antibodies from a patient sample, under conditions conducive to binding of the antibody to the peptide, and
iii. detecting the presence of antibodies bound to said peptide attached to said solid support.

To determine the efficacy of the subject peptides in detecting anti-SCoV antibodies, the peptides are tested for their immunoreactivity with serum/plasma specimens obtained from patients with clinically diagnosed SARS. Such SARS-specific sera were provided by National Taiwan University Hospital in Taipei and by the SARS emergency hospital in Beijing, Xiaotangshan Hospital.

The peptides can be readily synthesized using standard techniques, such as the Merrifield solid phase method of synthesis and the myriad of available improvements on that technology^{24,25}. The peptides can also be made using recombinant DNA technology. As such, nucleic acid molecules encoding the SCoV antigenic peptides and immunologically functional analogues of the SCoV antigenic peptides and compliments thereof are encompassed by the invention. Vectors, especially expression vectors, comprising the nucleic acid molecules of the invention are also encompassed by the invention. Host cells containing the vectors of the invention are also encompassed by the invention. The invention also encompasses methods of producing the SCoV antigenic peptides and immunologically functional analogues of the SCoV antigenic peptides comprising incubating a host cell containing an expression vector comprising a nucleic acid molecule encoding an SCoV antigenic peptide and/or immunologically functional analogue of an SCoV antigenic peptide under such conditions that the SCoV antigenic peptide and/or immunologically functional analogue of an SCoV antigenic peptide is expressed.

The peptide compositions of the present invention are a significant advantage over the virus, virus-lysate and recombinant antigens of the prior art. It has been found that the SCoV is genetically stable and the conserved across isolates.⁹ An approach using controlled and well-defined immunogens rather than complex immunogens has substantial advantages. The quality of antigens produced by a chemical process or recombinant DNA technology can be better controlled and, as a result, reproducibility of the test results can be assured. No biohazardous materials are used in the manufacture of peptide antigens, reducing risks and eliminating the need for expensive biological containment. As site-specific epitopes presenting high molar concentrations of selected epitopes, signal-to-noise ratio is boosted and sensitivity is heightened by the peptide compositions, while specificity is optimized due to the reduction of undesirable cross-reactive epitopes. In a preferred embodiment, the peptides of the invention are synthesized. The use of synthetic peptides eliminates the false-positive results caused by the presence of antigenic materials originating from host cells and from recombinant protein expression systems that may be co-purified with SCoV viral and recombinant proteins. For example, sera from normal patients may have antibodies to SCoV host cells, or to recombinant Escherichia coli, yeast or baculovirus which are then cross-reactive with the antigenic materials used in diagnostic tests based on the biologically-derived antigens. The high specificity of the synthetic peptide-based immunoassay of the present invention makes it useful for differentiating infections caused by different viruses having similar clinical symptoms, e.g., RSV or influenza versus SARS. Another advantage of the synthetic peptides is cost. The costs for producing immunoassays having synthetic peptides are relatively low in comparison to tests using virally and recombinantly-produced antigens because smaller amounts of peptides are required for each test procedure, and because the expense of preparing peptides is relatively low¹⁵⁻¹⁷.

The peptide compositions prepared in accordance with the present invention can be used to detect SCoV antibodies by using them in an antigenically effective amount as the antigen (e.g. solid phase immunosorbent) in immunoassay test kits. In accordance with the present invention, any suitable immunoassay format can be used with the subject peptides. Such formats are well known to the ordinarily skilled artisan and have been described in many standard immunology manuals and texts, see for example, by Harlow et al.²⁶. The include, among other well-known immunoassay formats, an enzyme-linked immunoadsorbent assay (ELISA), an enzyme immunodot assay, an agglutination assay, an antibody-peptide-antibody sandwich assay, a peptide-antibody-peptide sandwich assay. In a preferred embodiment, the immunoassay is an ELISA using a solid phase coated with the above-identified peptide compositions. ELISA techniques are well known in the art.

The immunoassays and/or diagnostic kits of the present invention are used to screen patient samples (e.g., body fluids and tissues) for the presence of anti-SCoV-reactive antibody. Immunoassays containing one or more of these peptides, or segments thereof, are useful to identify antibodies induced by infection or by vaccination. Thereby, such tests can be used as diagnostic tools to aid in diagnosis of SARS, to monitor antibody and viral antigen expression during SCoV infection and thereby determine correlations between the presence of specific antibodies and the prognosis of SARS in patients, and in epidemiological surveys and/or to monitor the effectiveness of a vaccination program.

Preferably the kit of this invention is an ELISA or an agglutination test kit for detection of SCoV antibodies and thereby diagnosis of SARS. For an ELISA or an agglutination test kit, the kit contains (a) a container (e.g., a 96-well plate) having a solid phase coated with one or more of the peptides of the invention; (b) a negative control sample; (c) optionally, a positive control sample; (d) specimen diluent and (e) antibodies to species-specific (e.g., human) IgG, or protein A, protein G or protein A/G recombinants known to be reactive with all types or subtypes of immunoglobulins from multiple species, which protein is labeled with a reporter molecule. If the reporter molecule is an enzyme, then the kit also contains a substrate for said enzyme.

In an exemplified use of the subject kit, a patient sample to be tested is diluted in sample diluent if necessary and then contacted with one or more peptides of the invention for a time and under conditions for any antibodies, if present, to bind to the peptide contained in the container. After removal of unbound material (e.g., by washing with phosphate buffered saline), the secondary complex is contacted with labeled antibodies to species-specific IgG or labeled protein A, protein G, or protein A/G. These antibodies or proteins A, G or A/G bind to the secondary complex to form a tertiary complex and, since the second antibodies or proteins A, or G or A/G are labeled with a reporter molecule, when subjected to a detecting means, the tertiary complex is detected. The reporter molecule can be an enzyme, radioisotope, fluorophore, bioluminescent molecule, chemiluminescent molecule, biotin, avidin, streptavidin or the like. For ELISA the reporter molecule is preferably an enzyme.

The examples serve to illustrate the present invention and are not to be used to limit the scope of the invention.

### Example 1

### Site-specific Serology for Mapping SCoV Protein Antigenic Epitopes

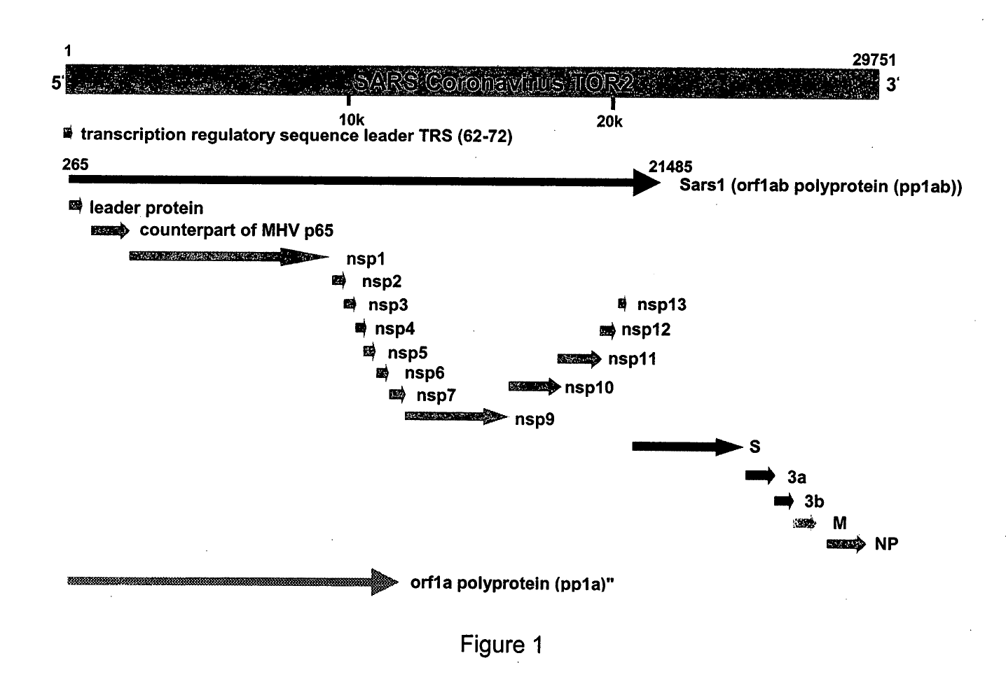

The first genomic sequence of a SCoV was for Tor2, isolated in Toronto²³. The deduced protein sequences of Tor2 as shown in FIG. 1 were used to align the structural protein sequences of all other SCoV isolates which are available from the GenBank database. Such alignments allow for the identification of isolate-to-isolate mutations which may have occurred in the individual proteins.

The information obtained from Tor2 was used to design candidate peptide antigens as shown in FIGS. 2 and 3, with peptide codes from 3171 on, for identification and location of antigenic sites within SCoV structural proteins for the development of SARS diagnostic tests for antibody detection and vaccines.

Over 200 short and long peptides with sequences derived from the SCoV Spike(S), Membrane(M), and Nucleocapsid (N) proteins as shown in FIGS. 2 and 3 were synthesized. Although predicted secondary structures were considered in the design of these overlapping peptides, emphasis was placed on an empirical selection of candidate antigenic peptides by their immunoreactivities with sera from patients infected by SCoV.

Short peptides comprising about 20 acids were synthesized with overlaps of about 10 residues across the entire amino acid sequences for the M, N, and S proteins. These were produced by automated peptide synthesizers (AccuChem, Lexington, Ky.) for the mapping of continuous epitopes.

Longer peptides comprising from about 25 up to about 100 amino acids were synthesized using Applied BioSystems Peptide Synthesizer Models 430A, 431 and 433. These were made to correspond to large regions of the S and N proteins and were useful to present longer processions of continuous epitopes and for greater ability to present conformational epitopes.

Each peptide was produced by an independent synthesis on a solid-phase support, with Fmoc protection for the terminus and side chain protecting groups of trifunctional amino acids. Completed peptides were cleaved from the solid support and side chain protecting groups removed by 90% trifluoroacetic acid. Synthetic peptide preparations were characterized for correct composition by Matrix-Assisted Laser Desorption Time-Of-Flight (MALDTOF) Mass Spectrometry and by Reverse Phase HPLC.

Antigenicities of the synthesized peptides of varied lengths were tested for initial reactivity profiles with sera from 1) patients clinically diagnosed and confirmed for SCoV infection at National Taiwan University Hospital in Taipei and at the SARS emergency hospital in Beijing, Xiaotangshan Hospital, 2) from those of healthy blood donors with zero prevalence rate for SCoV infection, collected in 2000 from Florida blood banks, 3) from patients with other known viral infections such as HCV, and 4) from patients having sera containing interfering substances, by the standard ELISA method described below in Example 2. Those with specific reactivities to sera from confirmed SARS patients were selected for further analyses. Since the volume of the sera available for use were of very small quantity, mostly in the range of less than 100 μL, we used two SARS samples, JPR and CZS, of which we had larger volumes to carry out the initial screens for SCoV-specific reactivities.

### Example 2

### ELISA Assay Method

The wells of 96-well plates were coated separately for 1 hour at 370 with 2 μg/mL of SCoV S, M, and N protein-derived peptides or mixtures thereof using 100 μL per well in 10 mM NaHCO₃ buffer, pH 9.5 unless noted otherwise.

The peptide-coated wells were incubated with 250 μL of 3% by weight of gelatin in PBS in 37° C. for 1 hour to block non-specific protein binding sites, followed by three washes with PBS containing 0.05% by volume of TWEEN 20 and dried. Patient sera positive for SCoV-reactive antibody by IFA and control sera were diluted 1:20, unless otherwise noted, with PBS containing 20% by volume normal goat serum, 1% by weight gelatin and 0.05% by volume TWEEN 20. One hundred microliters of the diluted specimens were added to each of the wells and allowed to react for 60 minutes at 37° C.

The wells were then washed six times with 0.05% by volume TWEEN 20 in PBS in order to remove unbound antibodies. Horseradish peroxidase-conjugated goat anti-human IgG was used as a labeled tracer to bind with the SCoV antibody/peptide antigen complex formed in positive wells. 100 μL of the peroxidase-labeled goat anti-human IgG at a pretitered optimal dilution and in 1% by volume normal goat serum, 0.05% by volume TWEEN 20 in PBS, was added to each well and incubated at 37° C. for another 30 minutes.

The wells were washed six times with 0.05% by volume TWEEN 20 in PBS to remove unbound antibody and reacted with 100 μL of the substrate mixture containing 0.04% by weight 3′,3′,5′,5′-Tetramethylbenzidine (TMB) and 0.12% by volume hydrogen peroxide in sodium citrate buffer for another 15 minutes. This substrate mixture was used to detect the peroxidase label by forming a colored product. Reactions were stopped by the addition of 100 μL of 1.0M H₂SO₄ and absorbance at 450 nm (A₄₅₀) determined.

### Example 3

### Identification of Antigenic Peintides Derived from SCoV M and S Proteins

A large collection of overlapping peptides of lengths varying from 20 to 76 residues with amino acid sequences derived from SCoV Tor2 M and S proteins were designed for empirical testing by positive sera.

In another method for epitope identification, specific features of predicted secondary structure in peptides known to be antigenic are used to select peptides which are synthesized and tested for antigenicity. However, in practice, theoretical prediction of antigenic features by algorithm has proven less useful for immunoassay development than empirical analysis for serological reactivity across the entire sequence of an antigenic protein by experiment²².

Initially, the antigenicities of SCoV M and S protein-derived peptides, each with an amino acid sequence derived from the corresponding positions shown in FIGS. 2 and 3, were determined with serum samples from two confirmed SARS CoV-infected patients (coded as JPR and CZS in Tables 2 and 3) for which we had access to 2 mL serum each, diluted at 1:20, in the ELISA format described in Example 2. Panels of serum/plasma samples non-reactive for SCoV, coded as NSP1, 2, 3, 4, 5, 6 and 7 for the M peptide (Table 2), and NSP 4, 6, 7, 8, 14 and 15 for S peptide (Table 3) were used in parallel runs to determine the background reactivities for the respective peptides, so as to assure the low background and specificity of the selected peptides.

After analysis of the serological data, one peptide designated as 3301 (SEQ ID NO. 5) derived from the M protein (Table 2) and one designated as 3180c derived from the S protein (SEQ ID NO. 2) (Table 3) were identified as having antigenicities with anti-SCoV antibodies. The parallel testings of peptides on the wells coated at either 1, 2, 5 or 10 μg/mL confirmed the specific reactivities for both the M (SEQ ID NO. 5) and the S (SEQ ID NO. 2) peptides for serum JPR and a preferential reactivity of serum CZS with the M peptide (SEQ ID NO. 5). The absorbancies shown in Table 2 and 3 also established the optimal plate coating concentrations for 3301 and 3180c as 2 μg/mL.

Testing of the selected M and S peptides each at the optimal 2 μg/mL coating condition with a larger panel of sera from patients with confirmed SARS and from the seroconversion panels of two patients exposed to SCoV with serial bleed dates of days 1-116 and 0-97 further demonstrated SARS anti-SCoV reactivity profiles for the M and S peptides (Table 4).

TABLE 2
Antigenicity of SCoV M protein-derived peptide
by ELISA at varied peptide coating concentrations

| | A_{450 nm} | | | |
|---|---|---|---|---|
| | p3301 coating concentration (SEQ ID NO. 5) | | | |
| Sample ID | 1 μg/mL | 2 μg/mL | 5 μg/mL | 10 μg/mL |
| Blank | 0.046 | 0.045 | 0.075 | 0.059 |
| NRC | 0.056 | 0.052 | 0.085 | 0.068 |
| SARS-JPR | 0.479 | 0.606 | 0.476 | 0.315 |
| SARS-CZS | 0.307 | 0.405 | 0.346 | 0.253 |
| NSP-1 | 0.053 | 0.052 | 0.083 | 0.067 |
| NSP-2 | 0.051 | 0.049 | 0.078 | 0.064 |
| NSP-3 | 0.050 | 0.059 | 0.077 | 0.064 |
| NSP-4 | 0.060 | 0.058 | 0.087 | 0.075 |
| NSP-5 | 0.055 | 0.059 | 0.089 | 0.077 |
| NSP-6 | 0.059 | 0.058 | 0.095 | 0.075 |
| NSP-7 | 0.051 | 0.049 | 0.081 | 0.148 |

TABLE 3
Antigenicity of SCoV S protein-derived peptide by
ELISA at varied peptide coating concentrations

| | A_{450 nm} | | | |
|---|---|---|---|---|
| | 3180c coating concentration (SEQ ID NO. 2) | | | |
| Sample ID | 1 μg/mL | 2 μg/mL | 5 μg/mL | 10 μg/mL |
| Blank | 0.045 | 0.044 | 0.045 | 0.045 |
| NRC | 0.065 | 0.059 | 0.122 | 0.097 |
| SARS-JPR | 0.670 | 0.723 | 1.599 | 1.473 |
| SARS-CZS | 0.089 | 0.096 | 0.162 | 0.198 |
| NSP-4 | 0.070 | 0.059 | 0.095 | 0.092 |
| NSP-6 | 0.097 | 0.074 | 0.177 | 0.132 |
| NSP-7 | 0.072 | 0.062 | 0.160 | 0.197 |
| NSP-8 | 0.065 | 0.057 | 0.062 | 0.074 |
| NSP-14 | 0.061 | 0.066 | 0.177 | 0.175 |
| NSP-15 | 0.053 2, | 0.058 | 0.123 | 0.120 |

TABLE 4
Antigenicities of SCoV S, M and N protein-derived peptides p3180c,
p3301, and 3190b with an enlarged sera panel for sensitivity evaluation

| | A_{450 nm} | | |
|---|---|---|---|
| Sample ID | 3180c | 3301 | 3190b |
| Blank | 0.051 | 0.048 | 0.047 |
| NRC | 0.096 | 0.069 | 0.070 |
| SARS-T3 | 1.617 | 0.856 | 1.467 |
| SARS-T4 | 1.052 | 1.060 | 1.506 |
| SARS-T5 | 1.862 | 0.495 | 2.553 |
| SARS-T6 | 0.289 | 0.844 | 1.180 |
| SARS-T7 | 1.131 | 0.540 | 2.474 |
| SARS-B1 | 0.185 | 0.255 | 0.576 |
| SARS-B3 | 0.680 | 0.260 | 0.785 |
| SARS-B9 | 0.172 | 0.177 | 0.881 |
| SARS-B10 | 0.198 | 0.174 | 0.161 |
| SARS-JPR | 1.484 | 0.780 | 1.558 |
| SARS-CZS | 0.107 | 0.593 | 0.818 |
| Day 0 SARS-CSG 4/22 | 0.107 | 0.069 | 0.070 |
| Day 6 SARS-CSG 4/28 | 0.161 | 0.168 | 0.194 |
| Day 16 SARS-CSG 5/8 | 0.702 | 0.080 | 1.259 |
| Day 27 SARS-CSG 5/19 | 0.465 | 0.497 | 2.516 |
| Day 116 SARS-CSG | 0.343 | >3 | 2.066 |
| 8/26 | | | |
| Day 0 SARS-LFJ 5/9 | 0.212 | 0.083 | 0.181 |
| Day 11 SARS-LFJ 5/20 | 2.681 | 1.340 | >3 |
| Day 17 SARS-LFJ 5/26 | 2.159 | 2.651 | >3 |
| Day 38 SARS-LFJ 6/16 | 2.526 | 2.718 | >3 |
| Day 97 SARS-LFJ 8/14 | 2.691 | 2.449 | >3 |

### Example 4

### Identification of Antigenic Peptides Derived from SCoV N Protein

A large collection of overlapping peptides of lengths varying from 20 to 81 residues with amino acid sequences derived from SCoV Tor2 N protein were designed for empirical testing by positive sera.

Initially, the antigenicities of SCoV N protein-derived peptides, each with an amino acid sequence derived from the corresponding positions shown in FIG. 2, were determined with serum samples from two confirmed SCoV-infected patients (coded as JPR and CZS in Table 5) for which we had access to 2 mL serum each, diluted at 1:2.5, in the ELISA format described in Example 2. A panel of serum/plasma samples non-reactive for SCoV, coded as NSP-2, 3, and 12 (Table 5), were used in parallel runs to determine the background reactivities for the respective peptides, so as to assure the low background and specificity of the selected peptides.

After analysis of the serological data, eight peptides derived from the N protein, designated as 3187b, 3187, 3189a, 3189b, 3189, 3190a, 3190b, and 3190 (SEQ ID NOS. 7-13 and 15, respectively) were identified as having antigenicities with anti-SCoV antibodies (Table 5), with peptides of the 3190 series (SEQ ID NOS. 12,13,15) having the most consistent reactivities.

TABLE 5
Antigenicities of N protein derived peptides (A₄₅₀ₙₘ by ELISA)

| | 3187b | 3187 | 3189a | 3189b | 3189 | 3190a | 3190b | 3190 |
|---|---|---|---|---|---|---|---|---|
| | (ID NO | (ID NO | (ID NO | (ID N0 | (ID N0 | (ID | (ID N0 | (ID N0 |
| Sample ID | 7) | 8) | 9) | 10) | 11) | N012) | 13) | 15) |
| Blank | 0.046 | 0.047 | 0.048 | 0.050 | 0.047 | 0.049 | 0.046 | 0.047 |
| NRC | 0.120 | 0.125 | 0.067 | 0.065 | 0.065 | 0.063 | 0.065 | 0.104 |
| NSP-2 | 0.073 | 0.077 | 0.074 | 0.73 | 0.084 | 0.069 | 0.069 | 0.066 |
| NSP-3 | 0.071 | 0.076 | 0.071 | 0.63 | 0.061 | 0.062 | 0.058 | 0.061 |
| NSP-12 | 0.079 | 0.081 | 0.082 | 0.081 | 0.077 | 0.130 | 0.146 | 0.101 |
| SARS-JPR (1/2.5 dil) | 0.093 | 0.125 | 0.303 | 0.246 | 0.227 | 0.634 | 0.732 | 0.579 |
| SARS-CZS (1/2.5 dil) | 0.298 | 0.303 | 0.131 | 0.122 | 0.116 | 0.235 | 0.323 | 0.248 |

Parallel ELISA testing of antigenic N peptide 3190b (SEQ ID NO.13) coated on the wells at 1, 2, 5 and 10 μg/mL, with the SARS and normal sera further confirmed the specific reactivity of that N peptide for sera JPR and CZS (Table 6).

TABLE 6
Antigenicity of SCoV N protein-derived peptide 3190b by ELISA
at various peptide coating concentrations

| | A_{450 nm} | | | |
|---|---|---|---|---|
| | 3190b coating concentration (SEQ ID NO. 13) | | | |
| Sample ID | 1 μg/mL | 2 μg/mL | 5 μg/mL | 10 μg/mL |
| Blank | 0.063 | 0.054 | 0.047 | 0.050 |
| NRC | 0.067 | 0.070 | 0.069 | 0.071 |
| SARS-JPR (⅙ dil) | 0.316 | 0.323 | 0.332 | 0.306 |
| SARS-CZS (½ dil) | 0.272 | 0.458 | 0.493 | 0.436 |
| NSP-1 | 0.070 | 0.063 | 0.060 | 0.060 |
| NSP-2 | 0.073 | 0.065 | 0.066 | 0.064 |
| NSP-20 | 0.107 | 0.102 | 0.100 | 0.099 |

Testing of selected N peptide 3190b at the 2 μg/mL coating condition with a larger panel of sera from patients with confirmed SARS and from the seroconversion panels of the two subjects exposed to SCoV further demonstrated SARS anti-SCoV reactivity profiles for the N peptide (Table 4).

### Example 5

### Immunologically Functional Peptide Analogues having Size or Charge Modifications by Extensions or Deletions or by Addition of Lys-Lys-Lys Residues at N Terminus Are Useful in SARS Immunoassay Formulation

Two types of immunologically functional peptide analogues having 1) size or 2) charge modifications were used to illustrate their antigenicities with anti-SCoV antibodies and thus their usefulness in assay formulation.

SARS patient serum JPR was used to assess the peptide analogues varied in size from the parent S-derived peptide 3180c (SEQ ID NO. 2). Elimination of 10 amino acids from its N-terminus resulted in a peptide designated as 3180b (SEQ ID NO. 1), or extension at its N-terminus with 15 amino acids resulted in a peptide designated as 3081 (SEQ ID NO. 4) (Table 7). The three peptides were used in ELISAs at plate coating concentrations of 2 μg/mL. Both peptide analogues demonstrated significant immunoreactivities with SARS patient serum JPR, comparable to that of parent peptide 3180c (Table 7). The negative reactivities of all three peptides with normal serum or plasma samples (NSP 9-11) further supported their specific reactivities with the anti-SCoV antibodies present in patient serum JPR, thus establishing their status as immunologically functional analogues.

TABLE 7
Antigenicities of size modified analogues of S protein derived antigenic peptide p3180c

| | A_{450 nm} | | |
|---|---|---|---|
| Sample ID | 3180b | 3180c | 3180 |
| Blank | 0.044 | 0.044 | 0.045 |
| NRC | 0.071 | 0.104 | 0.053 |
| SARS-JPR | 1.000 | 1.134 | 0.452 |
| SARS-CZS | 0.063 | 0.084 | 0.06 |
| NSP-SP9 | 0.068 | 0.069 | 0.051 |
| NSP-10 | 0.059 | 0.061 | 0.058 |
| NSP-11 | 0.061 | 0.057 | 0.063 |

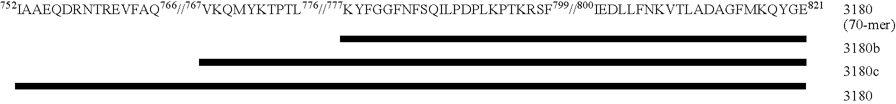

SARS patient samples JPR and CZS then were used to evaluate charge-modified peptide analogues to parent S and M peptides 3180c and 3301 (SEQ ID NOS. 2 and 5). Three consecutive lysine residues were added to the N-termini of peptides 3180c and 3301 resulting in peptides with designations as 3K3180c and 3K3301 (SEQ ID NOS. 4 and 6). ELISA plates were coated with each of these peptides at a concentration of 2 μg/mL and sera were used for the ELISA testing at a final 1:50 dilution, so as to illustrate the improved antigenicities of these charged analogues. As shown in Table 8, reactivity to JPR was significantly increased when comparing peptide 3301 to its further charged analogue 3K3301 (SEQ ID NO. 6), and in the comparison of peptide 3180c to its charged analogue 3K3180c (SEQ ID NO. 4). Reactivity to CZS was significantly increased for 3K3301 (SEQ ID NO. 6) when compared to its parent 3301, while low background reactivities with normal serum/plasma samples were maintained. Thus, the further charged analogues retained high specificities for anti-SCoV antibodies despite increased sensitivities and are immunologically functional analogues to their parent peptides.

TABLE 8
Antigenicities of charge modified (+3K) analogues of the S
and M protein-derived peptides 3180c and 3301

A_{450 nm}

| Sample ID | 3301 | 3K3301 | 3180c | 3K3180c |
|---|---|---|---|---|
| Blank | 0.048 | 0.045 | 0.048 | 0.044 |
| NRC | 0.059 | 0.053 | 0.063 | 0.072 |
| SARS-JPR | 0.287 | 0.452 | 0.319 | 0.696 |
| SARS-CZS | 0.168 | 0.261 | 0.057 | 0.068 |
| NSP-6 | 0.059 | 0.052 | 0.062 | 0.079 |
| NSP-7 | 0.054 | 0.052 | 0.056 | 0.055 |
| NSP-12 | 0.067 | 0.058 | 0.072 | 0.097 |
| NSP-30 | 0.058 | 0.056 | 0.075 | 0.075 |

### Example 6

### Peptide Compositions Having a Mixture of Antigenic SCoV Peptides in Assay Formulation Enhances Sensitivity

Although early detection of SARS is done by laboratory criteria such as RT-PCR assays using molecular probes and by clinical criteria such as elevated body temperature, non-productive cough, etc.², an antibody detection assay that is both sensitive and specific is desirable for serological surveillance. In developing our SARS antibody detection assays for serosurveillance and diagnosis, assay specificity had been stressed as a high priority. High specificity is a requisite of an acceptable SARS antibody test so as not to misdiagnose patients for unnecessary isolation, and to avoid the unnecessary implementation of emergency public health measures to contain an outbreak. As shown in previous examples, two selected SCoV peptides 3301 and 3180c both demonstrated reactivities with high stringency for SARS patient sera. However, an acceptable immunoassay for serosurveillance and diagnosis must also have high sensitivity. Therefore, mixtures of the antigenic S and M peptides were evaluated as antigens for complimentary sensitivity for antibody detection. Peptides 3180c and 3301 were coated alone at the previously established optimal 2 μg/mL concentration (Tables 2 and 3) and compared to peptide mixtures coated at respective concentrations of 1+1 μg/mL, 1.5+3 μg/mL, and 2+2 μg/mL by evaluation with SARS patient sera JPR and CZS diluted 1:20. For the 1.5+3 μg/mL mixed peptide coating concentration, additional SARS patient sera T3, T4, T5, T6 and T7 were also evaluated for subtle differences in assay sensitivity. As shown in Table 9, SARS patient samples CZS, T5 and T6 were found to be below the detection level with wells coated with either peptide 3180c or 3301 alone. Significant increases in antibody reactivities for these sera were observed when they were tested in wells coated with the mixed peptides, as shown by the areas marked in gray in Table 9. Moreover, the use of the peptide mixtures did not result in loss of specificity as shown by the low background reactivities of the mixtures for the normal sera. Thus sensitivity was improved and specificity was retained for the assay formulation using a mixture of complimentary peptide antigens as the solid phase antigen adsorbent.

TABLE 9
Improved sensitivity by using a mixture of antigenic peptides

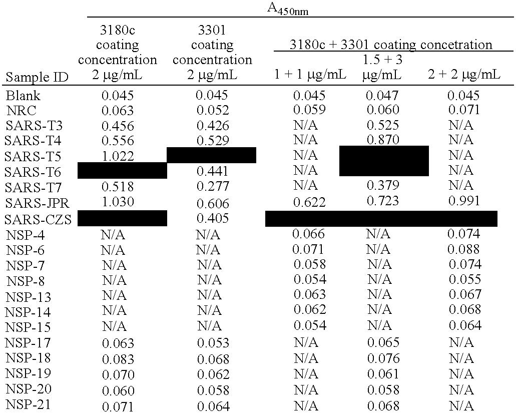

### Example 7

### Evaluation of SARS Enzyme Immunoassay in Infected, Random Blood Donor, and Other Non-SCoV Infected Populations, in a Large Scale Analysis

Characterization of confirmed SARS patient sera. Sera from patients confirmed as having SCoV infection were shown to have antibody titers of from 1:200 to 1:800 against SCoV as determined by an immunofluorescence assay (IFA) on cells from a monkey kidney cell line infected with SCoV.

Sera from patients infected with other viruses and normal sera. Sera obtained prior to 2000 from patients with other viral infections unrelated to SARS were well documented by serological markers. A panel of 672 sera from normal blood donors was obtained from a Florida Blood bank in 2000. The seroprevalence rate for reactivity to SCoV in these sera panels, collected at least three years prior to the report of any known SARS cases were used to evaluate the specificity of the SARS ELISA.

Analysis by a mixed peptide-based SARS SCoV ELISA. SCoV peptide ELISAs were conducted on 96-well microtiter plates coated with a mixture of SCoV S (3180c) and M (3301) peptides at 0.5 μg/mL and 3 μg/mL respectively, and with sera diluted 1:20 by the method described in Example 2.

Criteria for interpretation: Significant reactivity in the ELISA format, i.e., the cutoff value, was scored by A₄₅₀ absorbances which were greater than the mean A₄₅₀ plus six standard deviations of the distribution of sera from the normal population.

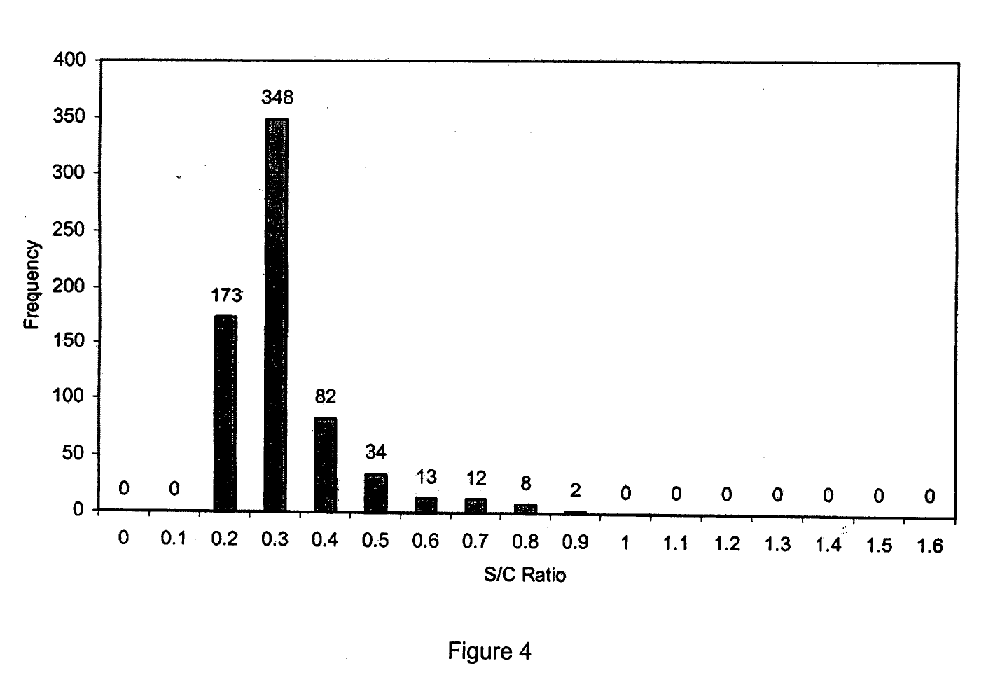

Results: The samples from a panel of 672 normal plasma and serum samples with a presumed zero seroprevalence rate were tested at 1:20 dilutions to assess their respective reactivities in the mixed peptide SCoV ELISA. The normal donor samples gave a mean A₄₅₀ of 0.074±0.0342 (SD), establishing a cutoff value of A₄₅₀ 0.279. The distribution of the Signal to Cutoff (S/C) ratio for the normal sera is plotted as shown in FIG. 4 with the peak S/C ratio having a value of 0.3, with none of the samples showing positive reactivity. Thus, the specificity of this ELISA on the normal samples was 100% at the set cutoff value.

The SCoV ELISA was further evaluated for specificity by testing with a large panel of samples from patients with infections unrelated to SCoV, such as HIV-1, HIV 2, HCV, HTLV 1 μl, and syphilis, and with normal serum samples spiked with interference substances. These samples all tested negative by the SCoV ELISA, indicating further the high specificity of the mixed peptide test (Tables 10 and 11).

TABLE 10
Evaluation of the specificity of a mixed peptide SCoV ELISA for
samples of HCV, HIV, HTLV, and Syphilis-infected sera and normal sera

| Sample ID | A_{450 nm} | S/C ratio |
|---|---|---|
| Blank | 0.045 | 0.17 |
| NRC | 0.069 | 0.26 |
| Cutoff | 0.269 | 1.00 |
| HCV pnl-1 | 0.071 | 0.26 |
| HCV pnl-2 | 0.091 | 0.34 |
| HCV pnl-3 | 0.068 | 0.25 |
| HCV pnl-4 | 0.068 | 0.25 |
| HCV pnl-5 | 0.099 | 0.37 |
| HCV pnl-6 | 0.068 | 0.25 |
| HCV pnl-7 | 0.055 | 0.20 |
| HCV pnl-8 | 0.063 | 0.23 |
| HCV pnl-9 | 0.072 | 0.27 |
| HCV pnl-10 | 0.072 | 0.27 |
| HCV pnl-11 | 0.064 | 0.24 |
| HCV pnl-12 | 0.067 | 0.25 |
| HIV 1 pnl-1 | 0.144 | 0.54 |
| HIV 1 pnl-2 | 0.084 | 0.31 |
| HIV 1 pnl-3 | 0.048 | 0.18 |
| HIV 1 pnl-4 | 0.095 | 0.35 |
| HIV 1 pnl-5 | 0.072 | 0.27 |
| HIV 1 pnl-6 | 0.078 | 0.29 |
| HIV 1 pnl-7 | 0.054 | 0.20 |
| HIV 1 pnl-8 | 0.068 | 0.25 |
| HIV 1 pnl-9 | 0.087 | 0.32 |
| HIV 1 pnl-10 | 0.119 | 0.44 |
| HIV 1/2 pnl-1 | 0.061 | 0.23 |
| HIV 1/2 pnl-2 | 0.077 | 0.29 |
| HIV 1/2 pnl-3 | 0.076 | 0.28 |
| HIV 1/2 pnl-4 | 0.065 | 0.24 |
| HIV 1/2 pnl-5 | 0.077 | 0.29 |
| HIV 2 pnl-1 | 0.058 | 0.22 |
| HIV 2 pnl-2 | 0.068 | 0.25 |
| HIV 2 pnl-3 | 0.071 | 0.26 |
| HIV 2 pnl-4 | 0.070 | 0.26 |
| HIV 2 pnl-5 | 0.063 | 0.23 |
| HIV 2 pnl-6 | 0.066 | 0.25 |
| HIV 2 pnl-7 | 0.065 | 0.24 |
| HTLV I/II pnl-1 | 0.047 | 0.17 |
| HTLV I/II pnl-2 | 0.049 | 0.18 |
| HTLV I/II pnl-3 | 0.053 | 0.20 |
| HTLV I/II pnl-4 | 0.047 | 0.17 |
| HTLV I/II pnl-5 | 0.048 | 0.18 |
| HTLV I/II pnl-6 | 0.049 | 0.18 |
| HTLV I/II pnl-7 | 0.047 | 0.17 |
| HTLV I/II pnl-8 | 0.090 | 0.33 |
| HTLV I/II pnl-9 | 0.090 | 0.33 |
| HTLV I/II pnl-10 | 0.058 | 0.22 |
| HTLV I/II pnl-11 | 0.056 | 0.21 |
| HTLV I/II pnl-12 | 0.056 | 0.21 |
| Syphilis pnl-1 | 0.058 | 0.22 |
| Syphilis pnl-2 | 0.079 | 0.29 |
| Syphilis pnl-3 | 0.052 | 0.19 |
| Syphilis pnl-4 | 0.064 | 0.24 |
| Syphilis pnl-5 | 0.049 | 0.18 |
| Syphilis pnl-6 | 0.058 | 0.22 |

TABLE 11
Evaluation of the specificity of a mixed peptide SCoV
ELISA on samples with interfering substances

| | OD_{450 nm} by | |
|---|---|---|
| Sample ID | ELISA | S/C ratio |
| Blank | 0.045 | 0.17 |
| NRC | 0.066 | 0.25 |
| Cutoff | 0.266 | 1.00 |
| interference FD2-2312-1a | 0.047 | 0.17 |
| interference FD2-2312-1b | 0.061 | 0.23 |
| interference FD2-2312-1c | 0.046 | 0.17 |
| interference FD2-2312-1d | 0.061 | 0.23 |
| interference FD2-2312-2a | 0.087 | 0.32 |
| interference FD2-2312-2b | 0.056 | 0.21 |
| interference FD2-2312-2c | 0.052 | 0.19 |
| interference FD2-2312-3a | 0.051 | 0.19 |
| interference FD2-2312-3b | 0.050 | 0.19 |
| interference FD2-2312-3c | 0.054 | 0.20 |
| interference FD2-2312-3d | 0.054 | 0.20 |
| interference FD2-2312-3e | 0.044 | 0.16 |
| interference FD2-2312-4a | 0.045 | 0.17 |
| interference FD2-2312-4b | 0.062 | 0.23 |
| interference FD2-2312-4c | 0.070 | 0.26 |
| interference FD2-2312-4d | 0.086 | 0.32 |
| interference FD2-2312-4e | 0.053 | 0.20 |
| interference FD2-2312-5a | 0.050 | 0.19 |
| interference FD2-2312-5b | 0.051 | 0.19 |
| interference FD2-2312-5c | 0.052 | 0.19 |
| interference FD2-2312-5d | 0.048 | 0.18 |
| interference FD2-2312-5e | 0.050 | 0.19 |
| interference FD2-2312-6a | 0.051 | 0.19 |
| interference FD2-2312-6b | 0.056 | 0.21 |
| interference FD2-2312-6c | 0.055 | 0.20 |
| interference FD2-2312-6d | 0.052 | 0.19 |
| interference FD2-2312-6e | 0.057 | 0.21 |
| interference FD2-2312-7a | 0.047 | 0.17 |
| interference FD2-2312-7b | 0.047 | 0.17 |
| interference FD2-2312-7c | 0.053 | 0.20 |
| interference FD2-2312-7d | 0.056 | 0.21 |
| interference FD2-2312-7e | 0.050 | 0.19 |
| interference FD2-2312-8a | 0.055 | 0.20 |
| interference FD2-2312-8b | 0.049 | 0.18 |
| interference FD2-2312-8c | 0.049 | 0.18 |
| interference FD2-2312-8d | 0.050 | 0.19 |
| interference FD2-2312-8e | 0.052 | 0.19 |
| interference FD2-2312-9a | 0.054 | 0.20 |
| interference FD2-2312-9b | 0.059 | 0.22 |
| interference FD2-2312-9c | 0.051 | 0.19 |
| interference FD2-2312-9d | 0.053 | 0.20 |

(a: Normal serum,
b: Heparin,
c: EDTA,
d: ACD, and
e: CPDA-1)

TABLE 12
Evaluation of the sensitivity of the mixed peptide SCoV ELISA on
serum samples from SARS patients including two seroconversion panels

| Sample ID | A_{450 nm} | S/C ratio |
|---|---|---|
| Blank | 0.045 | 0.17 |
| NRC | 0.069 | 0.26 |
| Cutoff | 0.269 | 1.00 |
| SARS-JPR | 1.656 | 6.16 |
| SARS-CZS | 1.750 | 6.51 |
| SARS-T3 | 1.712 | 6.36 |
| SARS-T4 | 1.806 | 6.71 |
| SARS-T5 | 1.208 | 4.49 |
| SARS-T6 | 1.214 | 4.51 |
| SARS-T7 | 1.080 | 4.01 |
| SARS-B1 | 0.309 | 1.15 |
| SARS-B3 | 0.613 | 2.28 |
| SARS-B9 | 0.464 | 1.72 |
| Day 0 SARS-CSG 4/22 | 0.068 | 0.25 |
| Day 6 SARS-CSG 4/28 | 0.163 | 0.61 |
| Day 16 SARS-CSG 5/8 | 0.345 | 1.28 |
| Day 27 SARS-CSG 5/19 | 1.212 | 4.51 |
| Day 116 SARS-CSG 8/26 | >3 | >9.4 |
| Day 0 SARS-LFJ 5/9 | 0.119 | 0.44 |
| Day 11 SARS-LFJ 5/20 | 1.638 | 6.09 |
| Day 17 SARS-LFJ 5/26 | 2.447 | 9.10 |
| Day 38 SARS-LFJ −6/16 | 2.749 | 10.22 |
| Day 97 SARS-LFJ 8/14 | 2.600 | 9.67 |

Further serological analysis with additional sera obtained from infected SARS patients reconfirmed the efficacy of the mixed peptide SCoV ELISA as depicted in Table 12. All sera obtained from patients with confirmed SARS and samples shown to have antibody titers against SCoV as detected by IFA were found to be positive by the mixed peptide SCoV ELISA. Table 12 also shows an analysis of the reactivity status of two SARS patients with serial bleed dates ranging from days 0 to 116 for patient CSG and from days 0 to 97 for patient LFJ. Results from these pedigreed seroconversion panels indicated that detectable levels of anti-SCoV M and S antibodies appeared as early as 11 and 16 days upon infection (for patient LFJ and CSG respectively). Such antibodies persisted in high titers throughout a 100 day period (Table 12).

### Example 8

### Evaluation of SARS Enzyme Immunoassay in Populations Having Serological Reactivities to Other Viruses and Pneumonia-causing Pathogens Other then the SARS Coronavirus

The high specificity of the synthetic peptide-based SARS immunoassay should make it a useful serological assay for differentiating SARS from pneumonia caused by other pathogens.

The mixed peptide ELISA described and characterized above in Examples 6 and 7 was further evaluated on sera panels from National Taiwan University Medical School having samples from 1)10 patients naturally infected with influenza (two sequential bleeds per flu patient), 2) 10 patients with rubella, 3) eight with cytomegalovirus (CMV) infection, 4) nine with Epstein Barr Virus (EBV), 5) five infected with mycoplasma, a bacterial cause for the kind of atypical pneumonia caused by SCoV, and 6) pre- and post-vaccine bleeds from 16 patients given influenza vaccine. Also included were serial samples collected from three SARS patients on the indicated days. All samples were tested in duplicate. The data (Table 13) were negative for all patients infected with pneumonia-causing pathogens other than the SARs coronavirus and were positive on all but early bleed SARS patient samples.

In addition, human coronaviruses 229E and OC43 belong to a different serotype from the SARS coronavirus, and an immunoassay of adequate specificity for distinguishing SARS from other coronavirus respiratory infections should not have cross-reactivity to these other coronaviruses^{10,23}. The mixed peptide SARS ELISA did not detect reactivities for these coronaviruses in the 692 US blood donor samples presented in Example 7 (FIG. 4). There is a strong expectation for reactivity among a US population because, even among healthy young adults, the incidence of OC43 and 229E respiratory infections is as high as 8%27. The lack of detectable reactivities among a large number the US serum and plasma samples supports the specificity of the mixed peptide SARS ELISA.

The specificity and sensitivity results of this Example demonstrate that the mixed peptide SARS ELISA is an appropriate method for distinguishing pneumonia caused by SCoV from pneumonia caused by other pathogens.

In summary, a highly sensitive and specific SCoV antibody detection test in the simple, rapid, and convenient ELISA format was developed for the large scale application of serosurveillance for SARS. The test is based on a solid phase immunosorbent comprising antigenic synthetic peptides corresponding to segments of the SCoV M, S, and N proteins and immunologically functional analogues thereof, branched as well as linear forms, conjugates, and polymers. The immunoassay is suitable for use in combination with molecular probe-based or other virus detection systems. The high specificity of this peptide-based SCoV immunoassay system, provided by the high stringency imposed on the selection of the SCoV antigenic peptides, and the high sensitivity provided by the mixture of peptides having complementary site-specific epitopes, results in a test that is appropriate for national epidemiological surveys. Such tests can be used by countries suffering from SARS outbreak or suspecting the presence of SARS for look back epidemiology studies. Also, a highly specific immunoassay can be used to differentiate SCoV infection from diseases caused by unrelated respiratory viruses and bacteria. An immunoassay of the invention can eliminate the untoward over-reporting of SARS, reduce the number of patients in isolation, and reduce the other costs associated with emergency measures to contain disease transmission.

TABLE 13
Evaluation of the specificity of the mixed peptide SCoV ELISA on
samples from patients infected with influenza, rubella, CMV, EBV,
mycoplasma, or SCoV, and flu vaccine sera.

| | | S/C | |
|---|---|---|---|
| Sample ID | Serum description | ratio | Reactivity |
| UBIAP1-T1 | Flu, natural infection | 0.21 | neg |
| UBIAP1-T2 | Flu, natural infection | 0.20 | neg |
| UBIAP2-T1 | Flu, natural infection | 0.23 | neg |
| UBIAP2-T2 | Flu, natural infection | 0.22 | neg |
| UBIAP3-T1 | Flu, natural infection | 0.19 | neg |
| UBIAP3-T2 | Flu, natural infection | 0.19 | neg |
| UBIAP4-T1 | Flu, natural infection | 0.19 | neg |
| UBIAP4-T2 | Flu, natural infection | 0.30 | neg |
| UBIAP5-T1 | Flu, natural infection | 0.33 | neg |
| UBIAP5-T2 | Flu, natural infection | 0.20 | neg |
| UBIAP6-T1 | Flu, natural infection | 0.22 | neg |
| UBIAP6-T2 | Flu, natural infection | 0.21 | neg |
| UBIAP7-T1 | Flu, natural infection | 0.22 | neg |
| UBIAP7-T2 | Flu, natural infection | 0.23 | neg |
| UBIAP8-T1 | Flu, natural infection | 0.22 | neg |
| UBIAP8-T2 | Flu, natural infection | 0.21 | neg |
| UBIAP9-T1 | Flu, natural infection | 0.22 | Neg |
| UBIAP10-T1 | Flu, natural infection | 0.22 | Neg |
| 038074 | Rubella infection | 0.18 | Neg |
| 038076 | Rubella infection | 0.35 | Neg |
| 038092 | Rubella infection | 0.20 | Neg |
| 038093 | Rubella infection | 0.29 | Neg |
| 038095 | Rubella infection | 0.19 | Neg |
| 038099 | Rubella infection | 0.23 | Neg |
| 038100 | Rubella infection | 0.38 | Neg |
| 038109 | Rubella infection | 0.24 | Neg |
| 038112 | Rubella infection | 0.30 | Neg |
| 038115 | Rubella infection | 0.38 | Neg |
| 038009 | CMV infection | 0.38 | Neg |
| 038010 | CMV infection | 0.29 | Neg |
| 038013 | CMV infection | 0.32 | Neg |
| 038014 | CMV infection | 0.22 | Neg |
| 038043 | CMV infection | 0.29 | Neg |
| 038047 | CMV infection | 0.32 | Neg |
| 038065 | CMV infection | 0.52 | Neg |
| 038077 | CMV infection | 0.22 | Neg |
| 038045 | EBV infection | 0.26 | Neg |
| 038050 | EBV infection | 0.22 | Neg |
| 038068 | EBV infection | 0.33 | Neg |
| 038080 | EBV infection | 0.36 | Neg |
| 038082 | EBV infection | 0.22 | Neg |
| 038086 | EBV infection | 0.33 | Neg |
| 038087 | EBV infection | 0.25 | Neg |
| 038094 | EBV infection | 0.24 | Neg |
| 038105 | EBV infection | 0.22 | Neg |
| 036893 | Mycoplasma infection | 0.27 | Neg |
| 37066 | Mycoplasma infection | 0.19 | Neg |
| 036883 | Mycoplasma infection | 0.30 | Neg |
| 037893 | Mycoplasma infection | 0.52 | Neg |
| 037623 | Mycoplasma infection | 0.46 | Neg |
| Flu vac 1-T1 | Flu vaccinate pre-bleed | 0.28 | Neg |
| Flu vac 1-T2 | Flu vaccinate | 0.24 | Neg |
| Flu vac 8-T1 | Flu vaccinate pre-bleed | 0.23 | Neg |
| Flu vac 8-T2 | Flu vaccinate | 0.24 | Neg |
| Flu vac 11-T1 | Flu vaccinate pre-bleed | 0.23 | Neg |
| Flu vac 11-T2 | Flu vaccinate | 0.25 | Neg |
| Flu vac 12-T1 | Flu vaccinate pre-bleed | 0.20 | Neg |
| Flu vac 12-T2 | Flu vaccinate | 0.21 | Neg |
| Flu vac 13-T1 | Flu vaccinate pre-bleed | 0.22 | Neg |
| Flu vac 13-T2 | Flu vaccinate | 0.20 | Neg |
| Flu vac 14-T1 | Flu vaccinate pre-bleed | 0.21 | Neg |
| Flu vac 14-T2 | Flu vaccinate | 0.21 | Neg |
| Flu vac 19-T1 | Flu vaccinate pre-bleed | 0.25 | Neg |
| Flu vac 19-T2 | Flu vaccinate | 0.25 | Neg |
| Flu vac 20-T1 | Flu vaccinate pre-bleed | 0.23 | Neg |
| Flu vac 20-T2 | Flu vaccinate | 0.22 | Neg |
| Flu vac 21-T1 | Flu vaccinate pre-bleed | 0.22 | Neg |
| Flu vac 21-T2 | Flu vaccinate | 0.18 | Neg |
| Flu vac 22-T1 | Flu vaccinate pre-bleed | 0.27 | Neg |
| Flu vac 22-T2 | Flu vaccinate | 0.25 | Neg |
| Flu vac 23-T1 | Flu vaccinate pre-bleed | 0.31 | Neg |
| Flu vac 23-T2 | Flu vaccinate | 0.25 | Neg |
| Flu vac 24-T1 | Flu vaccinate pre-bleed | 0.25 | Neg |
| Flu vac 24-T2 | Flu vaccinate | 0.27 | Neg |
| Flu vac 25-T1 | Flu vaccinate pre-bleed | 0.29 | Neg |
| Flu vac 25-T2 | Flu vaccinate | 0.32 | Neg |
| Flu vac 26-T1 | Flu vaccinate pre-bleed | 0.37 | Neg |
| Flu vac 26-T2 | Flu vaccinate | 0.28 | Neg |
| Flu vac 29-T1 | Flu vaccinate pre-bleed | 0.27 | Neg |
| Flu vac 29-T2 | Flu vaccinate | 0.23 | Neg |
| Flu vac 30-T1 | Flu vaccinate pre-bleed | 0.25 | Neg |
| Flu vac 30-T2 | Flu vaccinate | 0.21 | Neg |
| SARS-1-T1 | SCoV infection 1 (May 1, 2003) | 1.10 | Pos |
| SARS-1-T2 | SCoV infection 4 (May 2., 2003) | 1.37 | Pos |
| SARS-1-T3 | SCoV infection 4 (May 5, 2003) | 1.61 | Pos |
| SARS-3-T1 | SCoV infection 3 (May 7, 2003) | 1.01 | Pos |
| SARS-3-T2 | SCoV infection 3 (May 10, 2003) | 1.57 | Pos |
| SARS-4-T1 | SCoV infection 4 (May 5, 2003) | 0.29 | Neg |
| SARS-4-T2 | SCoV infection 4 (May 9, 2003) | 2.50 | Pos |

Values are reported as the Signal/Cutoff ratio (S/C ratio) for the mean values from each sample.
Notations “T1” and “T2” indicate serial bleeds on the same patient.

### Example 9

### Serological Survey of Healthcare Workers

A prospective study was performed in Taiwan using the SARS SCoV ELISA comprising a mixture of two peptides, described in Example 7, to determine the presence of asymptomatic infection among primary healthcare workers in hospitals that treated SARS patients. Sera samples from 623 healthcare workers without symptoms who agreed to be tested for the presence of one or more antibodies to the SARS CoV, not all of whom were in direct contact with SARS patients, were collected at Ho Ping, Yang Ming, En Chu Kong and Hsin Tai Hospitals, about four weeks after the outbreaks were recognized. Ho Ping and Yang Ming Hospitals had admitted SARS patients prior to the recognition of SARS and prior to the implementation of control measures by healthcare workers. Subsequently, they experienced transmission to healthcare workers. The En Chu Kong and Hsin Tai facilities admitted patients with implementation of control measures. Neither of the latter hospitals had recorded transmission of SARS to healthcare workers.

Three asymptomatic cases were detected by the ELISA out of 383 samples from Ho Ping, and one out of the 50 from nursing aids at Yang Ming. These 4 positive samples, indicative of asymptomatic infection, were confirmed for seropositivity by IFA. None of the 190 sera from the two hospitals without nosocomial infection displayed seroconversion.

A preliminary survey with the peptide ELISA detected asymptomatic clusters of seroconversion among exposed healthcare workers in two Taiwan hospitals that also experienced nosocomial disease. In contrast, no seroconversion was found among the exposed healthcare workers from two hospitals that had no apparent disease transmission to healthcare workers. The finding of asymptomatic seropositive subjects indicated that the test will be useful in larger retrospective surveillance studies, that are needed to fully define the epidemiology and spectrum of disease.

### Example 10

### Evaluation of Peptide Composition Having A Mixture of S. M, and N Peptides

SCoV peptide-based ELISA was conducted as described in Example 7 except that the microtiter plate was coated with a mixture of S peptide 3180c at 0.5 μg/ml, M peptide 3301 at 3.0 μg/ml, and N peptide 3190b at 0.1 μg/ml. This three peptide ELISA was evaluated for sensitivity with the sera from confirmed SARS patients shown in Table 14. In addition to the routine 1:20 dilution if serum used in the other Examples, sera used in the three peptide ELISA were diluted even further (see second column of Table 14) prior to testing. The three peptide SARS ELISA detected reactivity in all of the confirmed samples thus demonstrating enhanced sensitivity.

Moreover, the use of the three peptide mixture did not result in loss of specificity. A specificity panel of 77 sera from the collection of pre-2000 US blood bank samples was tested on the three peptide ELISA. All were non-reactive. Thus specificity was retained with a mixture of three complimentary peptide antigens.

TABLE 14
Evaluation of the Sensitivity of a Three Peptide SCoV ELISA

| | Serum dil | | |
|---|---|---|---|
| | (in addition | | |
| Sample ID | to 1:20 dil) | A_{450 nm} | S/C ratio |
| Blank | | 0.037 | |
| NRC-1 | | 0.059 | |
| NRC-2 | | 0.051 | |
| Cutoff | | 0.259 | 1.00 |
| SARS-JPR | 1:6 | 0.592 | 2.29 |
| SARS-CZS | 1:3 | 0.570 | 2.20 |
| SARS-LFJ | 1:12 | 0.739 | 2.85 |
| SARS-T3 | 1:3 | 0.797 | 3.08 |
| SARS-T4 | 1:3 | 1.200 | 4.63 |
| SARS-T5 | 1:3 | 0.848 | 3.27 |
| SARS-T6 | 1:3 | 0.596 | 2.30 |
| SARS-T7 | 1:3 | 0.666 | 2.57 |

1. A method of detecting SCoV antibodies in a patient sample comprising:
a) contacting said patient sample with one or more SCoV antigenic peptides selected from the group consisting of SEQ ID NOS: 1, 5, 7, 9, and 12 or immunologically functional analogues thereof selected from the group consisting of SEQ ID NOS: 2, 3, 4, 6, 8, 10, 11, 13, 14, and 15 under conditions conducive to binding; and
b) measuring binding between said patient sample and said SCoV antigenic peptides or immunologically functional analogues thereof;
wherein detection of binding between said patient sample and said SCoV antigenic peptides or immunologically functional analogues thereof indicates the presence of SCoV antibodies in said patient sample.

2. The method of claim 1 wherein said one or more SCoV antigenic peptides or immunologically functional analogues thereof are attached to a solid phase prior to contact with said patient sample.

3. The method of claim 1 wherein said patient sample is selected from the group consisting of blood, serum, plasma, saliva, urine, mucus, fecal matter, and tissue extract.

4. The method of claim 1 wherein said SCoV antigenic peptides are SEQ ID NOs:2, 5, and 13.

5. A method of detecting SCoV antibodies in a patient sample comprising:
a) contacting said patient sample with one or more immunologically functional analogues of any of the SCoV antigenic peptides selected from the group consisting of SEQ ID NOS: 1, 5, 7, 9, and 12 under conditions conducive to binding, wherein said on or more immunologically functional comprises one or more of the following modifications when compared to said SCoV antigenic peptides:
i) a deletion of 10 amino acids or less at the N-terminus or C-terminus;
ii) an addition of 15 amino acids or less at the N-terminus or C-terminus;
iii) one or more conservative substitutions;
iv) an addition of a branched structure at the C-terminus;
v) covalent attachment to another moiety;
vi) an altered charge; and
vii) one or more conservative or non-conservative substitutions such that the sequence of said immunologically functional analogue is the sequence of a strain of SCoV other than the Tor2 isolate of SCoV; and
b) measuring binding between said patient sample and said immunologically functional analogues;
wherein detection of binding between said patient sample and said immunologically functional analogues indicates the presence of SCoV antibodies in said patient sample.

6. The method of claim 5 wherein said one or more SCoV antigenic peptides or immunologically functional analogues thereof are attached to a solid phase prior to contact with said patient sample.

7. The method of claim 5 wherein said patient sample is selected from the group consisting of blood, serum, plasma, saliva, urine, mucus, fecal matter, and tissue extract.

8. A peptide selected from the group consisting of SEQ ID NOS: 1-15.

9. A nucleic acid molecule that encodes a peptide of any of SEQ ID NOS:1-15 or a complement thereof.

10. A vector comprising a nucleic acid molecule of claim 9.

11. The vector of claim 10 that is an expression vector.

12. An immunologically functional analogue of an SCoV antigenic peptide of any one of SEQ ID NOS:1, 5, 7, 9, and 12 wherein said immunologically functional analogue comprises one or more of the following modifications when compared to the corresponding SCoV antigenic peptide:
a) a deletion of 10 amino acids or less at the N-terminus or C-terminus;
b) an addition of 15 amino acids or less at the N-terminus or C-terminus;
c) one or more conservative substitution;
d) an addition of a branched structure at the C-terminus;
e) covalent attachment to another moiety;
f) an altered charge; and
g) one or more conservative or non-conservative substitutions such that the sequence of said immunologically functional analogue is the sequence of a strain of SCoV other than the Tor2 isolate of SCoV.

13. A nucleic acid molecule that encodes a peptide of claim 12 or a complement thereof.

14. A vector comprising a nucleic acid molecule of claim 13.

15. The vector of claim 14 that is an expression vector.


SEQUENCE LISTING (15 sequences)

SEQ ID NO: 1 - protein, 45 aa, SARS Coronavirus
KYFGGFNFSQILPDPLKPTKRSFIEDLLFNKVTLADAGFMKQYGE

SEQ ID NO: 2 - protein, 55 aa, SARS Coronavirus
VKQMYKTPTLKYFGGFNFSQILPDPLKPTKRSFIEDLLFNKVTLADAGFMKQYGE

SEQ ID NO: 3 - protein, 58 aa, SARS Coronavirus
  MISC_FEATURE: Synthetic Peptide
KKKVKQMYKTPTLKYFGGFNFSQILPDPLKPTKRSFIEDLLFNKVTLADAGFMKQYGE

SEQ ID NO: 4 - protein, 69 aa, SARS Coronavirus
AAEQDRNTREVFAQVKQMYKTPTLKYFGGFNFSQILPDPLKPTKRSFIEDLLFNKVTLADAGFMKQYGE

SEQ ID NO: 5 - protein, 22 aa, SARS Coronavirus
MADNGTITVEELKQLLEQWNLV

SEQ ID NO: 6 - protein, 25 aa, SARS Coronavirus
  MISC_FEATURE: Synthetic Peptide
KKKMADNGTITVEELKQLLEQWNLV

SEQ ID NO: 7 - protein, 65 aa, SARS Coronavirus
QLPQGTTLPKGFYAEGSRGGSQASSRSSSRSRGNSRNSTPGSSRGNSPARMASGGGETALALLLL

SEQ ID NO: 8 - protein, 80 aa, SARS Coronavirus
HIGTRNPNNNAATVLQLPQGTTLPKGFYAEGSRGGSQASSRSSSRSRGNSRNSTPGSSRGNSPARMASGGGETALALLLL

SEQ ID NO: 9 - protein, 50 aa, SARS Coronavirus
AQFAPSASAFFGMSRIGMEVTPSGTWLTYHGAIKLDDKDPQFKDNVILLN

SEQ ID NO: 10 - protein, 65 aa, SARS Coronavirus
DLIRQGTDYKHWPQIAQFAPSASAFFGMSRIGMEVTPSGTWLTYHGAIKLDDKDPQFKDNVILLN

SEQ ID NO: 11 - protein, 80 aa, SARS Coronavirus
GRRGPEQTQGNFGDQDLIRQGTDYKHWPQIAQFAPSASAFFGMSRIGMEVTPSGTWLTYHGAIKLDDKDPQFKDNVILLN

SEQ ID NO: 12 - protein, 52 aa, SARS Coronavirus
KKDKKKKTDEAQPLPQRQKKQPTVTLLPAADMDDFSRQLQNSMSGASADSTQ

SEQ ID NO: 13 - protein, 66 aa, SARS Coronavirus
KHIDAYKTFPPTEPKKDKKKKTDEAQPLPQRQKKQPTVTLLPAADMDDFSRQLQNSMSGASADSTQ

SEQ ID NO: 14 - protein, 69 aa, SARS Coronavirus
  MISC_FEATURE: Syntheitc Peptide
KKKKHIDAYKTFPPTEPKKDKKKKTDEAQPLPQRQKKQPTVTLLPAADMDDFSRQLQNSMSGASADSTQ

SEQ ID NO: 15 - protein, 81 aa, SARS Coronavirus
DDKDPQFKDNVILLNKHIDAYKTFPPTEPKKDKKKKTDEAQPLPQRQKKQPTVTLLPAADMDDFSRQLQNSMSGASADSTQ